SEQUENCE LISTING (24 sequences)

SEQ ID NO: 1 - protein, 11 aa, synthetic construct
GRKKRRQRRRC

SEQ ID NO: 2 - protein, 12 aa, synthetic construct
DSLKLSQEYESI

SEQ ID NO: 3 - protein, 24 aa, synthetic construct
DMAEHMERLKANDSLKLSQEYESI

SEQ ID NO: 4 - protein, 11 aa, synthetic construct
VGDGGLFEKKL

SEQ ID NO: 5 - protein, 15 aa, synthetic construct
YRSRKYSSWYVALKR

SEQ ID NO: 6 - protein, 4 aa, synthetic construct
RGDS

SEQ ID NO: 7 - protein, 5 aa, synthetic construct
IKVAV

SEQ ID NO: 8 - protein, 15 aa, synthetic construct
KLTWQELYQLKYKGI

SEQ ID NO: 9 - protein, 6 aa, synthetic construct
RKKADP

SEQ ID NO: 10 - protein, 4 aa, synthetic construct
EEEE

SEQ ID NO: 11 - protein, 5 aa, synthetic construct
EIDPK

SEQ ID NO: 12 - protein, 5 aa, synthetic construct
DIDPK

SEQ ID NO: 13 - protein, 4 aa, synthetic construct
VVAA

SEQ ID NO: 14 - protein, 6 aa, synthetic construct
VVVAAA

SEQ ID NO: 15 - protein, 4 aa, synthetic construct
AAVV

SEQ ID NO: 16 - protein, 6 aa, synthetic construct
AAAVVV

SEQ ID NO: 17 - protein, 6 aa, synthetic construct
VVAAEE

SEQ ID NO: 18 - protein, 4 aa, synthetic construct
GGGG

SEQ ID NO: 19 - protein, 5 aa, synthetic construct
GGGGG

SEQ ID NO: 20 - protein, 4 aa, synthetic construct
RRRR

SEQ ID NO: 21 - protein, 4 aa, synthetic construct
KKKK

SEQ ID NO: 22 - protein, 13 aa, synthetic construct
RRRRGGGGIKVAV

SEQ ID NO: 23 - protein, 13 aa, synthetic construct
KKKKGGGGIKVAV

SEQ ID NO: 24 - protein, 9 aa, synthetic construct
ILKNLSRSR

(19) United States
(12) Patent Application Publication
(10) Pub. No.: US 2026/0035413 A1
(43) Pub. Date: Feb. 5, 2026

(54) SLIDING BIOACTIVE PEPTIDES FOR THERAPEUTIC DELIVERY

(71) Applicant: Northwestern University, Evanston, IL (US)

(72) Inventors: Samuel Isaac Stupp, Evanston, IL (US); Federico Lancia, Evanston, IL (US); Madison Elizabeth Strong, Evanston, IL (US); Matias Alberto Alvarez-Saavedra, Evanston, IL (US)

(21) Appl. No.: 19/259,540

(22) Filed: Jul. 3, 2025

Related U.S. Application Data
(60) Provisional application No. 63/667,862, filed on Jul. 5, 2024.

(51) Int. Cl. C07K 14/00; A61K 35/30; A61K 38/00; A61K 47/62

(52) U.S. Cl. CPC C07K 14/001; A61K 35/30; A61K 47/62; A61K 38/00

(57) ABSTRACT

Provided herein are systems including a peptide amphiphile backbone and a peptide that interacts non-covalently with the peptide amphiphile. The peptide is able to move or slide along the surface of the PA, and is therefore referred to as a “slider”. Interaction of the slider with the PA backbone improves bioactivity, stability, and prevents the slider against degradation.

### PRIORITY STATEMENT

This application claims priority to U.S. Provisional Patent Application No. 63/667,862, filed Jul. 5, 2024, the entire contents of which are incorporated herein by reference for all purposes.

### SEQUENCE LISTING

The text of the computer readable sequence listing filed herewith, titled “NWEST-42206-203_SQL.xml”, created Jul. 2, 2025, having a file size of 21,763 bytes, is hereby incorporated by reference in its entirety.

### BACKGROUND

In 2016, an estimated 27 million people globally were living with SCI at varying severities. The risk of mortality increases with SCI, with 3.8% of patients dying within the first year, and lifespan is significantly decreased. Because of the detrimental outcomes, particularly in the chronic injury, therapeutic intervention is highly anticipated and of great interest to many in the field. Due to the complex nature of the chronic injury, many avenues are being explored to restore function. Such approaches include, but are not limited to, cell transplantation, small molecule delivery, wearable and implantable devices, and biomaterial design. Unfortunately, strategies that show promise in the acute injury may not be successful in the chronic phases of SCI. For example, transplanted cells directly to the injury site often do not survive or are overcome by the inhibitory landscape. Additionally, small molecule and growth factor therapeutics are limited by their short half-life poor localization. As such, there remains a need for effective methods of treating spinal cord injury.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show molecular designs of E2 PAs and peptides. FIG. 1A shows the chemical structure of TATISP and TATDSLK peptides. FIG. 1B shows a schematic representation of the ISP-based slider system. FIG. 1C shows a schematic representation of the sliding mechanism. The strength of the non-covalent interaction between the charged peptide segment of the PA (e.g. EE) and the charged sequence (e.g. the positively charged sequence) of the slider peptide can be tailored to modify the mobility of the slider peptide on the nanofiber.

FIGS. 2A-2B show SAXS and WAXS of sliders on E2 PAs. FIG. 2A shows flow-cell SAXS measurements of E2 PAs with 0.25 mol % peptides. FIG. 2B shows flow-cell WAXS measurements of E2 PAs with 0.25 mol % peptides.

FIGS. 3A-3F show β-sheet formation of sliders. CD spectra of E2 fibers (solid gray trendline) compared with sliders (solid color trendlines) and soluble peptide (dashed color trendlines). FIG. 3A shows results for E2-TATISP, FIG. 3B shows results for E2-R4ISP, FIG. 3C shows results for E2-ISP, FIG. 3D shows results for E2-TATDSLK, FIG. 3E shows results for E2-R4DSLK, and FIG. 3F shows results for E2-DSLK.

FIG. 4 shows morphology of sliders. TEM images of E2 fibers alone, and E2 fibers with 6.25 mol % of ISP and DSLK peptides

FIG. 5 shows binding of peptides to E2-TAMRA fibers. Confocal microscopy images of sliders made with E2-TAMRA and AF-488 conjugated peptides. Scale bar=100 μm

FIG. 6 shows bundling of TATDSLK sliders at different TAT-DSLK concentrations. Confocal microscopy images of sliders made with E2-TAMRA and AF-488 conjugated TAT-DSLK at varying concentrations of 0.1 to 10 mol %. Scale bar=50 μm

FIGS. 7A-7F show binding of peptides to E2 PA fibers. FIG. 7A, FIG. 7B, and FIG. 7C show FRET of ISP-based sliders made with E2-TAMRA and AF-488 conjugated peptides. FIG. 7D, FIG. 7E, and FIG. 7F show FRET of DSLK-based sliders. Unlabeled E2 was used as a control for all slider systems.

FIGS. 8A-8D show mobility of ISP sliders. FIG. 8A shows FRAP trajectory of AF-488 conjugated ISP-based peptides bound to E2 PAs. FIG. 8B shows fluorescence recovery percentage of ISP-based peptides. FIG. 8C shows FRAP trajectory of TAMRA-conjugated E2 PAs within ISP-based slider systems. FIG. 8D shows Fluorescence recovery percentage of E2 PAs within ISP-based sliders. For FIG. 8B and FIG. 8D, one-way ANOVA with multiple comparisons was performed with α=0.05: **P<0.01. ****P<0.0001.

FIGS. 9A-9D show mobility of DSLK sliders. FIG. 9A shows FRAP trajectory of AF-488 conjugated DSLK-based peptides bound to E2 PAs. FIG. 9B shows fluorescence recovery percentage of DSLK-based peptides. FIG. 9C shows FRAP trajectory of TAMRA-conjugated E2 PAs within DSLK-based slider systems. FIG. 9D shows fluorescence recovery percentage of E2 PAs within DSLK-based sliders. For FIG. 9B and FIG. 9D, one-way ANOVA with multiple comparisons was performed with α=0.05: ****P<0.0001.

FIGS. 10A-10B show impact of bundling on mobility. FIG. 10A shows FRAP trajectory of AF-488 conjugated TATDSLK peptides at different concentrations bound to E2 PAs. FIG. 10B shows FRAP trajectory of TAMRA-conjugated E2 PAs within TATDSLK sliders at different concentrations.

FIGS. 11A-11B show rheology measurements of TAT sliders. FIG. 11A shows storage and loss modulus of sliders with 1 mM E2 PAs. FIG. 11B shows storage and loss modulus of sliders with 5 mM E2 PAs

FIG. 12 shows effect of CSPG and HSPG on primary cortical neuron growth cones. Representative fluorescent micrographs of primary cortical neurons treated for 48 hours with 1 μg/mL CSPG or HSPG; neurons were stained with phalloidin (green), PTPσ (red), β-III tubulin (gray) and DAPI (blue).

FIGS. 13A-13D show recovery of neurons on CSPG treated with TATISP or TATDSLK. (A) Representative fluorescent micrographs of primary cortical neurons seeded on either PDL or CSPG and treated with TATISP or TATDSLK peptides for 48 hours; neurons were stained with B-III tubulin (green) and DAPI (blue). (B) Quantification of longest neurite length (C) Quantification of average branch number per neuron. (D) Bar graph representing number of cells per field of view in each condition. For B-D, one-way ANOVA with multiple comparisons was performed with α=0.05: *P<0.05. ***P<0.001. ****P<0.0001.

FIG. 14 shows morphology of neurons on PDL or CSPG treated with TATISP or TATDSLK. Representative micrographs of primary cortical neurons seeded on either PDL or CSPG and treated with TATISP or TATDSLK peptides for 48 hours; neurons were stained with phalloidin (red), β-III tubulin (green) and DAPI (blue); scale bar=20 μm.

FIGS. 15A-15B show neurite crossing on CSPG spot assay treated with DSLK materials. FIG. 15A shows representative micrographs of primary cortical neurons seeded on CSPG spot and treated with DSLK-based peptides and sliders for five days; neurons were stained with β-III tubulin (green) and DAPI (blue), and CSPG was stained with CS56 (red). FIG. 15B shows a bar graph representing the number of neurite crossings per area; one-way ANOVA with multiple comparisons was performed with α=0.05: ****P<0.0001.

FIGS. 16A-16B show cell survival on CSPG spot assay treated with DSLK materials. FIG. 16A shows representative micrographs of primary cortical neurons seeded on CSPG spot and treated with DSLK-based peptides and sliders for five days; neurons were stained with β-III tubulin (green) and DAPI (blue), and CSPG was stained with CS56 (red). FIG. 16B shows a bar graph representing the number of cells surviving on CSPG spot.

FIGS. 17A-17B show neurite crossing on CSPG spot assay treated with TATISP or TATDSLK sliders. FIG. 17A shows representative micrographs of primary cortical neurons seeded on CSPG spot and treated with TATISP or TATDSLK sliders for five days; neurons were stained with β-III tubulin (green) and DAPI (blue), and CSPG was stained with CS56 (red). FIG. 17B is a bar graph representing the number of neurite crossings per area; one-way ANOVA with multiple comparisons was performed with α=0.05: *P<0.05, **P<0.01.

FIG. 18 shows brightfield images taken every 24 hours after primary cortical neurons were scratched with a pipette tip and treated with CSPG and TATISP peptide (top) or slider (bottom). The slider system prolongs bioactivity following mechanical damage.

FIG. 19 shows representative fluorescent micrographs of primary cortical neurons 72 hours after they were scratched with a pipette tip and treated with or without 1 μg/mL CSPG; neurons were stained with β-III tubulin (green), phalloidin (red), and DAPI (blue), and (top) CSPG was stained with CS56 (gray); (bottom) scratch area is outlined with white dashes; scale bar=50 μm. CSPG localizes to the scratch area.

FIG. 20 shows neurite crossing after mechanical damage and CSPG inhibition. Representative fluorescent micrographs of primary cortical neurons 72 hours after they were scratched with a pipette tip and treated with 1 μg/mL CSPG and TAT-based peptides (top) or sliders (bottom); neurons were stained with β-III tubulin (green), phalloidin (red), and DAPI (blue); scratch area is outlined with white dashes.

FIG. 21 shows association of TATDSLK sliders with N2a cells. Representative fluorescent micrograph of N2a cells treated with TATDSLK sliders for 72 hours; live cells were imaged with cholera toxin subunit B (CtxB, gray), E2 PA fibers were labeled with TAMRA (red), and TATDSLK was labeled with AF-488; scale bar=10 μm.

FIG. 22 shows cellular uptake of TATISP and TATDSLK materials after 72 hours. Representative fluorescent micrographs of N2a cells treated with TAT-based peptides or sliders for 72 hours; live cells were stained with CellTracker (blue) (top), peptides were labeled with AF-488 (green), and E2 PA fibers were labeled with TAMRA (red); scale bar=10 μm.

FIG. 23 shows network formation following CSPG and TAT-based material treatment. Representative brightfield micrographs of primary cortical neurons treated with CSPG and TAT-based peptides (top) or sliders (bottom).

FIG. 24 shows synaptic formation is enhanced with TAT-based sliders. Representative fluorescent micrographs of primary cortical neurons treated with CSPG and TAT-based peptides (top) or sliders (bottom) for 14 days; synapses were stained with PSD-95 (green) and synaptophysin (red), and nuclei were stained with DAPI (blue).

FIG. 25 shows the structure of the E2 PA and representative structures of slider systems containing the backbone E2 PA and CNTF mimetic sliders.

FIG. 26 shows confocal microscopy results confirming colocalization of backbone PA and CNTF sliders.

FIGS. 27A-27C show results of FRAP experiments conducted using CNTF slider systems. FIG. 27A shows fluorescence intensity values. FIG. 27B shows the mobile fraction from FRAP experiments. FIG. 27C shows RMSF data from atomistic molecular dynamic simulation. FIG. 27D shows results from SAXS experiments demonstrating stabilization of the nanofiber by the sliders. FIG. 27E shows images demonstrating that sliders stabilize the nanofibers and induce transition to a ribbon-like morphology with increased width compared to E2 nanofibers alone. Results are quantified in FIG. 27F.

FIG. 28 shows experimental simulation results demonstrating that the interaction of the slider with the PA backbone also allows the slider to enable a more bioactive conformation, displaying hydrophobic side chains towards the outside of the fiber.

FIG. 29A shows images of microglial phagocytosis using the R-CNTF slider Microglial cells were treated with no treatment, recombinant CNTF as a positive control, E2 alone, the R-CNTF slider, or the CNTF mimetic peptide alone (slider only). Results are quantified in FIG. 29B. FIG. 29C shows signaling dynamics of the CNTF-mimetic slider system assessed by Western blot. The top panel shows raw western blot results, the bottom panel quantifies results.

FIG. 30 shows the chemical structures of the R₄G₄IKVAV (SEQ ID NO: 22) and K₄G₄IKVAV (SEQ ID NO: 23) slider peptides, which are protonated at biological pH to enable electrostatic interactions with the backbone PA.

FIG. 31A shows FRET spectra of the material with various mol % of the RIKVAV sliders. The characteristic quenching decrease at 520 nm and excitation increase at ˜580 nm are present in all samples except the control. FIG. 31B shows FRET spectra of the material with various mol % of the KIKVAV sliders, showing similar quenching and excitation shifts.

FIG. 32 shows samples of the material prepared at various concentrations and visualized under a confocal microscope. The IKVAV slider component of the molecule is labeled with TAMRA and visualized in red. The samples all contain E2 at 1 mM and have various mol % concentrations of RIKVAV (R) or KIKVAV (K) sliders. E2 alone shows relatively no bundling (not shown).

FIG. 33 shows SEM images of peptide amphiphile (PA) materials prepared with varying slider concentrations. E2 fibers alone form a uniform, unbundled layer. E2 fibers with 10% RIKVAV slider show the formation of smaller, interwoven bundles with visible individual fibers. E2 fibers with 50% RIKVAV slider exhibit larger, densely packed bundles, with individual fibers still distinguishable.

FIG. 34 shows cryo TEM imaging of 1 mM E2 nanofibers with and without various RIKVAV concentrations.

FIGS. 35A-35B show FRAP analysis of slider mobility on E2 fibers at varying concentrations. FIG. 35A shows normalized FRAP recovery curves for arginine-based sliders (R1%, R5%, R10%, R50%, R100%). FIG. 35B shows FRAP recovery for lysine-based sliders (K1%, K5%, K10%, K50%, K100%).

FIGS. 36A-36F show SAXS and WAXS data of E2 fibers functionalized with RIKVAV (FIG. 36A, FIG. 36B) and KIKVAV (FIG. 36C, FIG. 36D) sliders at varying concentrations (0%, 5%, 10%, 50%). FIG. 36A and FIG. 36C show SAXS data showing scattered intensity vs.

scattering vector (q) for RIKVAV and KIKVAV systems. The slope in the low-q region is indicated in red, with values ranging from −2.2 to −2.8. The negative form factor peak is highlighted near q=0.073 A⁻. FIG. 36B and FIG. 26D show WAXS data highlighting a sharp peak at q=4.71 A⁻¹. FIG. 36E shows summarized SAXS data WAXS data showing scattered intensity (cm⁻¹) and slope values for E2 fibers with RIKVAV. FIG. 36F shows summarized data for E2 fibers with KIKVAV sliders.

FIGS. 37A-37C show melting temperature experimental results. FIG. 37A shows Fluorescent intensity of nile red intercalated into the fiber of various concentration samples to indicate the temperature of disassembly of fiber (Melting temperature). FIG. 37B shows the plotted Derivative of data in A. FIG. 37C shows the plotted melting temperature across various materials (derived from peak in plot B).

FIG. 38 shows fluorescence microscopy images of immunostained primary cortical neurons on coverslips coated with PDL, laminin, E2 , E2+R10%, and E2+R50%. Neurons were fixed at DIV2 and stained with DAPI (blue) and βTubb3 (red), and imaged with an EVOS microscope. Maximum neurite length was measured using the Neuroanatomy plugin in ImageJ and analyzed using a Kruskal-Wallis test, followed by post hoc Dunn's test for pairwise comparisons (*P<0.05, **P<0.01). Results are quantified in the bar graph.

FIG. 39 shows DIV3 mouse primary cortical neurons treated on DIV0 with either no treatment (NT) or 40 μM E2 , E2+R10%, and E2+R50%. Neurons were stained with βubb3 (gray), phalloidin (green), and DAPI (blue, top row; pink, bottom row). High resolution images outlined in red are shown in the bottom row, illustrating PA fiber staining in the DAPI channel.

FIGS. 40A-40C show live/dead assay of DIV8 hNPCs treated on DIV1 with 20 μM of material. FIG. 40A shows the ratio of live/total cells quantified under conditions shown in (FIG. 40C). FIG. 40B shows total cells quantified under conditions shown in (FIG. 40C). FIG. 40C shows representative micrographs of hNPCs stained with calcein (live marker, green) and ethidium homodimer-1 (dead marker, red). Data was analyzed by a one-way ANOVA post hoc Tukey test.

FIG. 41 shows SEM images of DIV1 hNPCS treated with 20 μM E2 or E2+IKVAV slider at 10 mol % or 50 mol % on DIV0. Imaging depicts interactions between cells (red arrow) and material (yellow arrow) shortly after treatment. High magnification images are outlined in red.

FIG. 42 shows SEM images of DIV8 hNPCS treated with 20 μM E2 or E2+IKVAV slider at 10 mol % or 50 mol % on DIV1. High magnification images are highlighted in red.

FIGS. 43A-43C show immunostaining micrographs of DIV4 hNPCS treated on DIV1 with 20 μM material. FIG. 43A shows representative images of each sample are shown. Nestin is stained in red, DAPI in blue, total integrin (aITGB1) in green, and phalloidin in red. FIG. 43B shows high magnification images with DAPI in blue and aITGB1 in green. FIG. 43C shows graph quantifying the mean intensity of aITGB1 channel of all cells imaged divided by the area analyzed. Data was analyzed by a one-way ANOVA post hoc Tukey test (*P<0.05). Scale bar=100 μm (A) and 25 μm (B).

FIG. 44 shows immunostaining micrographs of DIV8 hNPCS treated on DIV1 with 20 μM material. Representative images of each sample are shown. Nestin is in red, DAPI is in blue, Pax6 is in green, and βTubb3 is in white.

FIGS. 45A-45B show immunostaining micrographs of DIV8 hNPCS treated on DIV1 with 20 μM material. Nestin is in red, DAPI is in blue, Pax6 is in green, and βTubb3 is in white. FIG. 45A shows Nyquist images of cells shown in FIG. 16 to more clearly visualize neuron morphology. FIG. 45B shows a graph of maximum neurite length of neurons present in immunostaining images. Maximum neurite length was measured using the Neuroanatomy plugin in ImageJ and analyzed using a Kruskal-Wallis test, followed by post hoc Dunn's test for pairwise comparisons (***P<0.001, ****P<0.0001). Scale bar=100 μm.

### SUMMARY

In some aspects, provided herein are systems comprising a peptide amphiphile (PA) comprising a hydrophobic segment, a structural peptide segment, and a charged peptide segment; and a peptide that interacts non-covalently with the peptide amphiphile. The PA is also referred to as a backbone, and the peptide that interacts non-covalently with the PA is also referred to as a slider or a slider peptide. In some embodiments, the system comprises a nanofiber. For example, in some embodiments provided herein is a system wherein a plurality of PAs assemble to form a nanofiber, and slider peptides interact with the surface of the nanofiber.

In some embodiments, the peptide (i.e. the slider peptide) comprises a biomimetic sequence and a charged sequence, wherein the charged sequence interacts non-covalently with the charged peptide segment of the peptide amphiphile. In some embodiments, the charged sequence interacts electrostatically with the charged peptide segment. For example, in some embodiments the charged peptide segment is negatively charged and the charged sequence is positively charged.

In some embodiments, the charged sequence comprises 4 to 20 positively charged amino acids. In some embodiments, the charged sequence comprises 4 to 10 positively charged amino acids. For example, in some embodiments the charged sequence comprises 4 to 10 lysine and/or arginine residues.

In some embodiments, the charged sequence comprises a sequence having at least 80% identity to GRKKRRQRRRC (SEQ ID NO: 1). In some embodiments, the charged sequence comprises SEQ ID NO: 1.

In some embodiments, the biomimetic sequence comprises a growth factor mimetic sequence, a cytokine mimetic sequence, a laminin mimetic sequence, an integrin mimetic sequence, an intracellular sigma peptide (ISP) sequence, or a truncate thereof. In some embodiments, the biomimetic sequence comprises an intracellular sigma peptide (ISP) sequence; a ciliary neurotrophic factor (CNTF) mimetic sequence; a vascular endothelial growth factor (VEGF) mimetic sequence, a brain-derived neurotrophic factor (BDNF) mimetic sequence, a glial cell-derived neurotrophic factor (GDNF) mimetic sequence, a fibroblast growth factor 2 (FGF-2) mimetic sequence, or a netrin-1 mimetic sequence.

In some embodiments, the ISP sequence comprises DSLKLSQEYESI SEQ ID NO: 2. In some embodiments, the ISP sequence comprises DMAEHMERLKANDSLKLSQEYESI (SEQ ID NO: 3). In some embodiments, the CNTF mimetic sequence comprises VGDGGLFEKKL (SEQ ID NO: 4).

In some embodiments, the biomimetic sequence comprises the VEGF mimetic sequence KLTWQELYQLKYKGI (SEQ ID NO: 8), the BDNF mimetic sequence RKKADP (SEQ ID NO: 9), the GDNF mimetic sequence ILKNLSRSR (SEQ ID NO: 24), the FGF-2 mimetic sequence YRSRKYSSWYVALKR (SEQ ID NO: 5), the netrin-1 mimetic sequence EIDPK (SEQ ID NO: 11), the netrin-1 mimetic sequence DIDPK (SEQ ID NO: 12), RGDS (SEQ ID NO: 6), or IKVAV (SEQ ID NO: 7).

In some embodiments, the peptide is mobile on the peptide amphiphile.

In some embodiments, the system comprises 1% mol to 60% mol of the slider peptide relative to the moles of the backbone PA. For example, in some embodiments the system comprises 5% mol of the peptide (e.g. 5% of the peptide, by moles, relative to the moles of the backbone PA). In some embodiments, the system comprises about 1% mol, about 5% mol, about 10% mol, about 15% mol, about 20%, about 25% mol, about 30% mol, about 35% mol, about 40% mol, about 45% mol, about 50% mol, about 55% mol, or about 60% mol of the slider peptide. The amount of the slider peptide may depend on the precise slider peptide used, e.g. the biomimetic sequence present in the slider peptide. In some embodiments, the system comprises 1% to 10% mol of the peptide. In some embodiments, the system comprises 5% to 15% mol of the peptide. In some embodiments, the system comprises 5% to 50% mol of the peptide. In some embodiments, the system comprises 40% to 60% mol of the peptide. In some embodiments, the system comprises 5% by mol of the peptide. In some embodiments, the system comprises 50% mol of the peptide. For example, in some embodiment the system comprises 1%-60% mol of the peptide comprising the biomimetic sequence of SEQ ID NO: 7, 1%-10% mol of the peptide comprising the biomimetic sequence of SEQ ID NO: 4, or 5% to 10% mol of the peptide comprising the biomimetic sequence SEQ ID NO: 2 or SEQ ID NO: 3.

In some embodiments, the system further comprises cells. For example, in some embodiments the cells comprise neurons.

The systems herein find use in a variety of methods including cell culture (e.g. as a scaffold to promote the growth, health, differentiation, etc. of a cell, and in methods of treating a subject having an injury. In some embodiments, the injury comprises a central nervous system injury. In some embodiments, the injury comprises a spinal cord injury.

### DETAILED DESCRIPTION

Of the three main components of chronic SCI, the astroglial scar border is a major contributor to spinal cord regeneration failure. One of the most abundant molecules within this border is chondroitin sulfate proteoglycans (CSPGs). These large molecules are composed of a core protein that is decorated with one or more CS polysaccharide glycosaminoglycan (GAG) chains. The negative charge provided by the sulfation moieties allows them to interact with a positively charged region of protein tyrosine phosphatase (PTP) receptors found within the neuronal membrane. Specifically, type IIa PTPs, including PTPσ, leukocyte antigen receptor (LAR), and PTP δ, have been found to interact strongly with CSPGs. These receptors are enzyme-linked transmembrane proteins that have two cytosolic PTP domains, D1 and D2, that enact phosphatase activity. While the mechanism of inhibition is not well understood, the CS chains play a role in PTP binding and growth restriction. Additionally, accumulation in distal regions may inhibit plasticity in the chronic injury, thus requiring intervention that addresses synaptic plasticity.

Interestingly, a similar molecule, heparan sulfate proteoglycans (HSPGs) oppose the inhibitory effect of their CS counterparts. The main structural difference between CSPG and HSPG lies within the sulfation pattern of their GAG chains. CS chains consist of evenly distributed sulfated groups (one-two sulfates per disaccharide), whereas HS chains consist of clusters of highly sulfated (three sulfates per disaccharide) regions surrounded by low sulfated regions and variably spaced by non-sulfated region. The modular sulfation pattern of HSPGs may cause clustering of PTP receptors, resulting in regions of unevenly distributed phosphatase activity. Through this clustering, the catalytic D1 domain of one receptor may interact with another, thus blocking the phosphatase activity. A helix-loop-helix (HLH) structure known as the wedge domain is located right before the first catalytic D1 domain, and is thought to contribute to this association.

Herein, a wedge-mimetic peptide targeting PTPσ, referred to as intracellular sigma peptide (ISP), was used in a peptide amphiphile system. The ISP sequence was truncated to contain the 12 amino acid residues on the C-terminus: DSLKLSQEYESI. This truncated sequence is referred to herein as “DSLK”. Because this portion of the wedge domain differs by only two residues between PTPσ and LAR, it was hypothesized that that the truncated form would bind and inhibit both receptors. The added benefit of this truncation is that when bound to PA nanostructures, the shorter length may avoid enzymatic degradation since it will be protected by the larger PA fibers. Additional slider PAs were synthesized herein, including sliders using a ciliary neurotrophic factor (CNTF) mimetic sequence and an IKVAV sequence. For all sliders PAs generated, interaction of the slider peptide with the PA backbone improved biological effect compared to the slider peptide alone.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” used in connection with a quantity is inclusive of the stated value and has the meaning dictated by the context (for example, it includes at least the degree of error associated with the measurement of the particular quantity). The term “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C. then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “scaffold” refers to a material capable of supporting growth and differentiation of a cell.

As used herein, the term “supramolecular” (e.g., “supramolecular complex,” “supramolecular interactions.” “supramolecular fiber,” “supramolecular polymer.” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” (“PA”) refers to a molecule that includes a hydrophobic segment, a structural peptide segment, and a charged peptide segment. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment; and (3) a charged peptide segment. Such a peptide amphiphile is also referred to herein as a “backbone PA”, a “backbone”, or a “PA backbone”.

As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)—where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used interchangeably herein, the terms “structural peptide” or “structural peptide segment” refer to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural peptide segments of adjacent structural peptide segments. In some embodiments, nanofibers of peptide amphiphiles having structural peptide segments display linear or 2D structure when examined by microscopy and/or a-helix and/or β-sheet character when examined by circular dichroism (CD). In some embodiments, nanofibers of peptide amphiphiles having structural peptide segments with a sufficient propensity for forming β-sheet conformations display an ordered secondary structure, such as rigid β-sheet conformations.

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the a “negatively-charged peptide segment” or an “acidic peptide segment” refers to a peptide sequence (e.g. a charged peptide segment) of a peptide amphiphile that comprises one or more amino acid residues that have side chains displaying carboxylic acid side chains (e.g., Glu (E), Asp (D), or non-natural amino acids). A negatively-charged peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the term “positively-charged peptide segment” or a “basic peptide segment” refers to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A positively charged peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “treat,” “treatment.” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment.” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### 2. Systems and Methods

In some aspects, provided herein systems comprising a peptide amphiphile (PA) and a peptide that interacts non-covalently with the PA. The peptide amphiphile is also referred to as a “backbone” (e.g. a backbone, a backbone PA, a PA backbone). In some embodiments, the strength of the non-covalent interaction can be tuned such that the peptide is mobile, e.g. slides and moves along the peptide amphiphile. A system comprising a peptide amphiphile and a peptide interacting non-covalently with the PA is thus referred to as a “slider system”, or a “slider PA”. The peptide that interacts non-covalently with the PA is referred to as a “slider” or a “slider peptide”. In some embodiments, the peptide amphiphiles assemble into a nanofiber, and the slider peptides interact with the surface of the nanofiber.

In some embodiments, the mobility of the peptide on the backbone PA (e.g. the slider peptide) facilitates delivery of the peptide extracellularly and/or intracellularly. The mobility of the slider peptide may be tuned depending on the desired properties of the slider system. Generally speaking, mobility can be decreased by increasing the strength of the non-covalent interaction between the slider peptide and the PA, whereas mobility can be increased by decreasing the strength of the non-covalent interaction. This can be achieved by use of different charged sequences in the slider PA, which charged sequences interact with the charged peptide segment of the peptide amphiphile. For example, the slider PA may comprise a charged sequence with a comparatively strong positive charge, which interacts strongly with a negatively charged domain (e.g. EE, EEE, EEEE (SEQ ID NO: 10)) in the PA. In contrast, a reduced positive charge in the charged sequence will diminish the strength of this interaction, increasing the mobility of the slider peptide on the PA. Increased mobility (e.g. achieved by a comparatively low strength of the non-covalent interaction between the slider peptide and the backbone PA), facilitates increased detachment of the slider PA from the backbone and may be advantageous for predominantly intracellular delivery of the peptide. Decreased mobility, achieved by a comparatively high strength of the non-covalent interaction between the slider peptide and the backbone PA, facilitates a stronger interaction between the slider and the backbone PA.

The systems herein have several advantages over use of the peptide alone (e.g. without the backbone PA) or compared to systems involving covalent conjugation of the peptide to a PA, or co-assembly of the peptide with PA molecules. For example, in some embodiments the backbone PA stabilizes the slider, protects the slider from degradation, and/or allows the slider to display a more bioactive conformation compared to the soluble peptide alone or compared to co-assemblies of the PA and the peptide. The systems herein are also advantageous over co-assemblies of peptides and PAs, which rely on interactions between the beta-sheet forming region of the peptide amphiphiles to form nanostructures containing the peptide, resulting in less dynamic peptides (peptides that are not mobile, or are less mobile than the slider peptides herein) that are less available for interactions with cell receptors or targets, and/or less capable of crossing into the cell for intracellular effect. In contrast, the systems herein even with comparatively high strength of the non-covalent interactions between the slider peptide and the PA backbone, are sufficiently mobile to permit detachment of the slider peptide from the backbone PA, which is not feasible in co-assemblies.

The systems herein have several uses, including in regenerative medicine (e.g. central nervous system regeneration, spinal cord healing, brain healing), wound healing, intracellular and/or extracellular delivery of therapeutic peptides, biomaterials for cell culture and adhesion, and the like.

In some embodiments, the peptide amphiphiles and compositions of the embodiments described herein are synthesized using preparatory techniques well-known to those skilled in the art. preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles (e.g. the backbone PA) comprise a hydrophobic segment (i.e. a hydrophobic tail) linked to a peptide segment. In some embodiments, the peptide segment is as structural peptide segment. In some embodiments, the structural peptide segment is a hydrogen-bond-forming segment, or beta-sheet-forming segment. In some embodiments, the peptide segment comprises a charged segment (e.g., acidic segment, basic segment, zwitterionic segment, etc.). In some embodiments, the peptide segment further comprises linker or spacer segments for adding solubility, flexibility, distance between segments, etc. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers). In some embodiments, the PA comprises a linker segment that connects the charged sequence to the biomimetic sequence. For example, in some embodiments the linker comprises one or more glycine residues (e.g. G, GG, GGG, GGGG (SEQ ID NO: 18), GGGGG (SEQ ID NO: 19), etc.).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) to bury the lipophilic segment in their core. In some embodiments, the structural peptide undergoes intermolecular hydrogen bonding to form beta sheets that orient parallel to the long axis of the micelle. In some embodiments, the structural peptide displays weak intermolecular hydrogen bonding, resulting in a less rigid beta-sheet conformation within the nanofibers.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment (e.g. a negatively charged peptide segment). For example, in some embodiments, the acidic peptide comprises one or more (e.g., 1, 2, 3, 4, 5, 6, 7, or more) acidic residues (e.g. negatively charged residues) (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises E₂₋₄. For example, in some embodiments an acidic peptide segment comprises EE. In some embodiments, an acidic peptide segment comprises EEE. In other embodiments, an acidic peptide segment comprises EEEE (SEQ ID NO: 10).

In some embodiments, peptide amphiphiles comprise a basic peptide segment (e.g. a positively charged peptide segment). For example, in some embodiments, the acidic peptide comprises one or more (e.g., 1, 2, 3, 4, 5, 6, 7, or more) basic residues (e.g. positively charged residues) (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural peptide segment. In some embodiments, the structural peptide segment has a propensity for forming β-sheet conformations. In some embodiments, the structural peptide segment is a beta-sheet-forming segment. In some embodiments, the structural peptide segment is rich in one or more of H, I, L, F, V, G, and A residues. In some embodiments, the structural peptide segment comprises an alanine-and valine-rich peptide segment (e.g., VVAA (SEQ ID NO: 13), VVVAAA (SEQ ID NO: 14), AAVV (SEQ ID NO: 15), AAAVVV (SEQ ID NO: 16), or other combinations of V and A residues, etc.). In some embodiments, the structural peptide segment comprises 4 or more consecutive A and/or V residues, or conservative or semi-conservative substitutions thereto. In some embodiments, the structural peptide segment comprises V₂A₂ (SEQ ID NO: 13).

In some embodiments, peptide amphiphiles comprise a non-peptide spacer or linker segment. In some embodiments, the non-peptide spacer or linker segment is located at the opposite terminus of the peptide segment from the hydrophobic segment. In some embodiments, the spacer or linker segment provides the attachment site for another moiety or component on the peptide amphiphile. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH₂(O(CH₂)₂)₂NHCO(CH₂)₂CCH, etc.). In some embodiments, a spacer or linker further comprises additional bioactive groups, substituents, branches, etc. In some embodiments, the linker segment is a single glycine (G) residue.

Suitable peptide amphiphiles for use in the materials herein, as well as methods of preparation of PAs and related materials, amino acid sequences for use in PAs, and materials that find use with PAs, are described in the following patents: U.S. Pat. Nos. 9,044,514; 9,040,626; 9,011,914; 8,772,228; 8,748,569; 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural peptide segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural peptide segment (e.g., comprising VVAA (SEQ ID NO: 13)); and (c) a charged segment (e.g., comprising EE, EEE, EEEE (SEQ ID NO: 10), etc.).

The system further comprises a peptide that interacts non-covalently with the peptide amphiphile (e.g. the backbone PA). This peptide is referred to herein as a “slider peptide” or a “slider”. In some embodiments, the peptide (e.g. the slider peptide) comprises a biomimetic sequence and a charged sequence. In some embodiments, the charged sequence interacts non-covalently with the charged peptide segment of the backbone PA. For example, in some embodiments the charged peptide segment is negatively charged (e.g. an acidic peptide segment, such as EE, EEE, EEEE (SEQ ID NO: 10)) and the charged sequence is positively charged, resulting in the non-covalent interaction. In this context, the charge refers to the overall charge of the sequence or segment. A “positively charged” sequence in the slider PA does not necessarily indicate that each and every amino acid in the positively charged sequence is basic. In some embodiments, the charged sequence comprises 4 to 20 positively charged (e.g. basic) amino acids. For example, in some embodiments the charged sequence comprises 4 to 20, 4 to 19, 4 to 18, 4 to 17, 4 to 16, 4 to 15, 4 to 14, 4 to 13, 4 to 12, 4 to 11, 4 to 10, 4 to 9, or 4 to 8 positively charged amino acids. In some embodiments, the positively charged (e.g. basic) amino acids in the charged sequence of the slider peptide are selected from lysine, arginine, and histidine residues. In some embodiments, the positively charged amino acids in the charged sequence are lysine and/or arginine residues. For example, in some embodiments the charged sequence comprises 4 to 20 lysine and/or arginine residues, 4 to 15 lysine and/or arginine residues, 4 to 10 arginine and/or lysine residues, or 4 to 6 arginine and/or lysine residues. In some embodiments, the charged sequence comprises 4 arginine residues (RRRR (SEQ ID NO: 20)). In some embodiments, the charged sequence comprises 4 lysine residues (KKKK (SEQ ID NO: 21)).

In some embodiments, the charged sequence comprises one or more neutral amino acids, in addition to the positively charged amino acids. The relative number of positively charged and/or neutral amino acids can be adjusted/modified to achieve the desired strength of interaction between the charged sequence and the charged peptide segment of the backbone PA, thereby tuning the properties of the system herein including the mobility of the slider PA, the detachment of the slider PA from the backbone, etc. In some embodiments, the neutral amino acids are selected from serine, threonine, asparagine, glutamine, alanine, valine, leucine, isoleucine, methionine, phenylalanine, tyrosine, tryptophan, proline, glycine, and cysteine.

In some embodiments, the charged sequence comprises 4 to 20 total residues, and at least 70% of the residues are positively charged (e.g. basic). In some embodiments, the charged sequence comprises 4 to 20 total residues, and at least 80% of the residues are positively charged (e.g. basic). In some embodiments, the charged sequence comprises 4 to 20 total residues, and at least 90% of the residues are positively charged (e.g. basic).

In some embodiments, the charged sequence comprises a transactivator of transcription (TAT) sequence. For example, in some embodiments the charged sequence comprises a sequence having at least 80% identity to GRKKRRQRRRC (SEQ ID NO: 1). For example, in some embodiments the charged sequence comprises a sequence having at least 90% identity to GRKKRRQRRRC (SEQ ID NO: 1). In some embodiments, the charged sequence comprises SEQ ID NO: 1. In some embodiments, the charged sequence is SEQ ID NO: 1.

The term “biomimetic sequence” as used herein refers to a sequence that mimics a biological activity or effect of a given moiety. For example, a “growth factor mimetic sequence” mimics a biological activity or effect of that growth factor. The biomimetic sequence is thus considered to be bioactive. In some embodiments, the biomimetic sequence is mimics the activity or effect of a moiety involved in the body's response to central nervous system injury (e.g. damage to the brain or the spinal cord). In some embodiments, the biomimetic sequence is a growth factor mimetic sequence (including neurotrophic growth factors), a cytokine mimetic sequence, a laminin mimetic sequence, an integrin mimetic sequence, an intracellular sigma peptide (ISP) sequence, or a truncate thereof.

Protein tyrosine phosphatase (PTP) receptors, including PTPσ, leukocyte antigen receptor (LAR), and PTP δ, have been found to interact strongly with CSPGs. In some embodiments, the biomimetic sequence comprises a wedge-mimetic peptide targeting PTPσ, referred to as intracellular sigma peptide (ISP), or a truncate thereof. ISP comprises the sequence DMAEHMERLKANDSLKLSQEYESI (SEQ ID NO: 3). In some embodiments, the biomimetic sequence comprises SEQ ID NO: 3. A slider peptide including the charged sequence of SEQ ID NO: 1 (e.g. the TAT sequence) and the biomimetic sequence of SEQ ID NO: 3 (e.g. the ISP sequence) is referred to herein as “TATISP”. In some embodiments, the biomimetic sequence comprises a sequence having at least 80% identity to SEQ ID NO: 3.

In some embodiments, the biomimetic sequence comprises a truncated ISP sequence. For example, in some embodiments the biomimetic sequence comprises a truncated ISP sequence containing at least 8 consecutive amino acids present in SEQ ID NO: 3. In some embodiments, the truncated ISP sequence contains at least 8, at least 9, at least 10, at least 11, or at least 12 consecutive amino acids present in SEQ ID NO: 3. In some embodiments, the biomimetic sequence comprises DSLKLSQEYESI (SEQ ID NO: 2). This truncated sequence is referred to herein as “DSLK”. A slider peptide including the charged sequence of SEQ ID NO: 1 and the biomimetic sequence of SEQ ID NO: 2 is referred to herein as “TATDSLK”. Without wishing to be bound by theory, because the wedge domain differs by only two residues between PTPσ and LAR, a truncated ISP sequence (e.g. SEQ ID NO: 2) may bind to and inhibit both receptors, resulting in improved activity compared to the full length ISP sequence (SEQ ID NO: 3). Furthermore, when bound to PA backbones, the shorter length sequence may avoid enzymatic degradation since it will be protected by the larger PA fibers.

In some embodiments, the biomimetic sequence comprises a ciliary neurotrophic factor (CNTF) mimetic sequence. CNTF is a the interleukin-6 family of cytokines, and is a factor that supports the survival and function of nerve cells. In some embodiments, the CNTF mimetic sequence comprises VGDGGLFEKKL (SEQ ID NO: 4). In some embodiments, the CNTF mimetic sequence comprises a sequence having at least 80% identity to SEQ ID NO: 4.

In some embodiments, the biomimetic sequence comprises a growth factor mimetic sequence. In some embodiments, the growth factor mimetic sequence is a vascular endothelial growth factor (VEGF) mimetic sequence, a brain-derived neurotrophic factor (BDNF) mimetic sequence, a glial cell-derived neurotrophic factor (GDNF) mimetic sequence, a fibroblast growth factor 2 (FGF-2) mimetic sequence, or a netrin-1 mimetic sequence.

In some embodiments, the VEGF mimetic sequence comprises KLTWQELYQLKYKGI (SEQ ID NO: 8). In some embodiments, the BDNF mimetic sequence comprises RKKADP (SEQ ID NO: 9). In some embodiments, the GDNF mimetic sequence comprises ILKNLSRSR (SEQ ID NO: 24). In some embodiments, the FGF-2 mimetic sequence comprises YRSRKYSSWYVALKR (SEQ ID NO: 5). In some embodiments, the netrin-1 mimetic sequence comprises EIDPK (SEQ ID NO: 11) or DIDPK (SEQ ID NO: 12).

In some embodiments, the biomimetic sequence comprises RGDS (SEQ ID NO: 6). In some embodiments, the biomimetic sequence comprises IKVAV (SEQ ID NO: 7).

In some embodiments, the biomimetic sequence comprises a sequence having at least 80%, at least 85%, at least 90%, or at least 95% identity with any of SEQ ID Nos: 2, 3, 4, 5 8, 9, 11, 12, or 24.

In some aspects, the system is a nanostructure, such as a nanofiber. For example, in some embodiments the system is a nanofiber comprising the backbone PA and the slider PA on the surface of the nanofiber. In some embodiments, the system comprises cells. For example, in some embodiments the system comprises neurons. In some embodiments, the system is a cell culture dish or plate containing cells, a cell culture medium, and the slider PA. In some embodiments, the system is a scaffold.

The systems herein find use in a variety of techniques, including cell culture and methods of treating injury. In some embodiments, the systems herein are used in methods of treating a disease, disorder, condition, or injury in a subject. The systems may be incorporated into a composition (e.g. ap pharmaceutical composition) for use in the methods herein. For example, in some embodiments the systems and compositions herein are used to promote neuronal growth, maturation, and/or signaling in a subject. In some embodiments, the subject has a disease or condition that causes neuronal injury and/or death, and the compositions provided herein improve one or more symptoms of the disease or condition by promoting neuronal growth (e.g. neurite outgrowth, synaptogenesis), maturation, and/or signaling in the subject. In some embodiments, the subject has received an injury that causes formation of a glial scar, and the systems and compositions herein promote neural growth (e.g. neurite growth, synaptogenesis) in spite of the presence of the glial scar which otherwise typically inhibits such recovery processes.

In some embodiments, the systems and compositions herein are used for methods of treatment or prevention of nervous system injury in a subject. For example, the PAs and nanofibers described herein may be used in methods for treatment of prevention of injury to the central nervous system (CNS), including the brain and the spinal cord, or the peripheral nervous system (PNS), including the nerves and ganglia outside of the brain and spinal cord. In some embodiments, the PAs and nanofibers described herein may be used for treatment or prevention of injury to the CNS or PNS in a subject. In some embodiments, the injury is a spinal cord injury. The spinal cord injury may be cervical, lumbar, thoracic, sacral, or any combination thereof. In some embodiments, the injury is a brain injury.

The injury may be a traumatic injury. A traumatic injury refers to an injury caused by trauma, for example trauma such as that caused by an automobile accident, a fall, violence, sports injury, surgical injury, and the like.) For example, the system and compositions described herein may be used for the treatment of traumatic central nervous system injury (e.g. traumatic spinal cord injury, traumatic brain injury (TBI). Alternatively, the injury may be a non-traumatic injury. For example, the injury may be a non-traumatic injury to the CNS (e.g., the brain and/or the spinal cord) or the PNS caused by, for example, cancer, multiple sclerosis, inflammation, arthritis, spinal stenosis, tumors, blood loss, stroke, and the like.

The systems or compositions described herein may be provided to a subject at any suitable point following injury (e.g. CNS injury) to treat the injury. For example, the composition may be provided to the subject within 24 hours of the injury (e.g. within 24 hours, within 12 hours, within 10 hours, within 9 hours, within 8 hours, within 7 hours, within 6 hours, within 5 hours, within 4 hours, within 3 hours, within 2 hours, or within 1 hour from injury. In some embodiments, the composition may be provided to the subject after a duration longer than 24 hours has passed following injury or diagnosis of injury.

The composition may be administered in any suitable amount, depending on factors including the age of the subject, weight of the subject, severity of the injury, and the like. The composition may be administered in combination with other suitable treatments for injury or preventative measures to prevent the severity of the injury from worsening.

In some embodiments, the systems or compositions herein are formulated for delivery to a subject. In some embodiments, the systems or compositions are formulated for parenteral administration (e.g. by injection). In some embodiments, the system or composition is applied directly to the site of the injury. For example, the system or composition may be applied topically directly to the site of the injury. As another example, the system or composition is injected at the site of the injury.

In some embodiments, the systems and composition herein are used in cell culture methods. For example, further disclosed herein are scaffolds (e.g. hydrogels) comprising the slider PA systems herein. The scaffolds may comprise a nanofiber of self-assembled peptide amphiphiles, at least a portion of the peptide amphiphiles comprising: a hydrophobic tail, a structural peptide segment, a charged peptide segment, and one or more slider peptides interacting with the charged peptide segment. The scaffolds described herein are capable of supporting growth and differentiation of a cell, including neurite outgrowth, synaptogenesis, and the like. Accordingly, the scaffolds may be in methods for culturing cells (e.g. neurons) or organoids (e.g. neural organoids, spinal cord organoids, etc). The methods for culturing cells or organoids comprise contacting the cells or organoids with a scaffold as described here. In some embodiments, the scaffold may be used as a coating for any desired cell culture tool (tissue culture plate, petri dish, glass slide, etc.).

Cells or organoids cultured on the scaffolds disclosed herein may demonstrate improved characteristics compared to cells or organoids cultured in the absence of the disclosed scaffolds. For example, cells or organoids may demonstrate improved differentiation, increased synaptogenesis, increased neurite outgrowth, improved maturation and/or improved long term viability compared to cells or organoids cultured in the absence of the disclosed scaffolds. In some embodiments, the scaffolds may be used in methods of culturing neuronal cells (e.g. neurons). In some embodiments, neurons cultured on the scaffolds provided herein display enhanced synaptogenesis and functional maturation (e.g. neurite outgrowth, axonal projections, improved electrical activity (e.g. cell signaling) etc.). In some embodiments, the scaffolds may be used in methods of culturing neural organoids, neurospheroids, and the like.

### EXPERIMENTAL

### Example 1

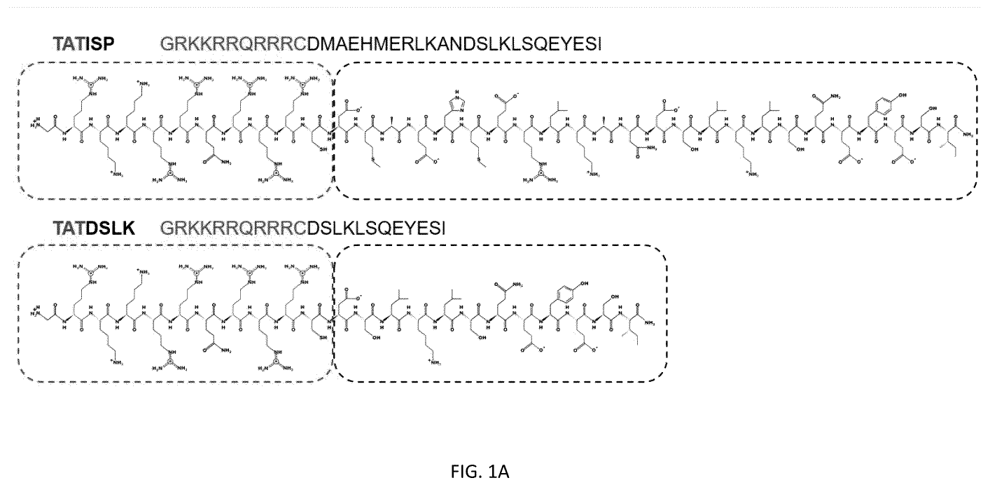

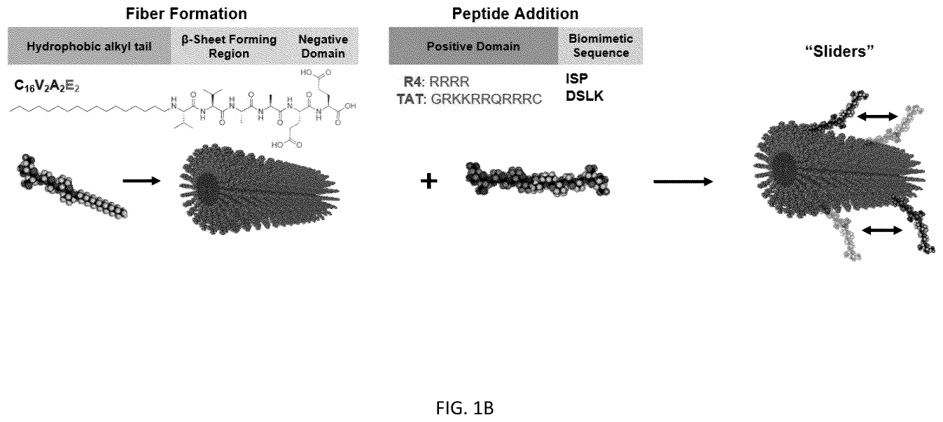

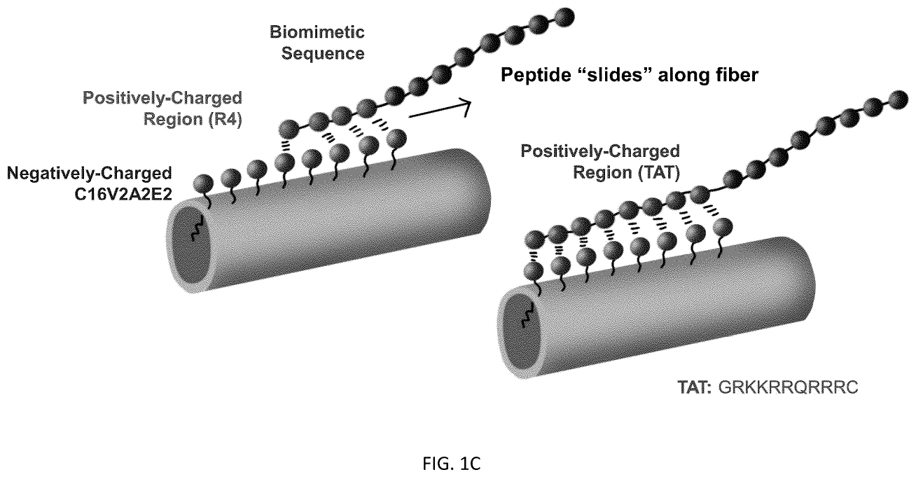

Spinal cord injury (SCI) is a traumatic event that often results in lifelong paralysis and dysfunction. The biological environment within the chronic injury is highly complex and prevents regeneration of spared neuronal tissue. Chondroitin sulfate proteoglycans (CSPGs) within the glial scar that surrounds the injury site are major contributors to the chemical inhibition of nerve cell regrowth. A soluble peptide called intracellular sigma peptide (ISP), which targets an intracellular region of a specific CSPG receptor, protein tyrosine phosphatase σ (PTPσ), was investigated for use in peptide amphiphile nanostructures herein. Peptide amphiphile (PA) nanostructures that interact noncovalently with either the ISP peptide or a modified form, DSLK, to help enhance the bioactivity, were developed and studied. The supramolecular fibers provide physical support to regenerating axons in the form of a scaffold while prolonging the delivery of the ISP and DSLK peptides. The ISP and DSLK peptides are conjugated with the positively charged transactivator of transcription (TAT) sequence to promote internalization into the cell. The positive residues are capable of electrostatically interacting with negatively charged PA fibers. This noncovalent binding decouples peptide signal mobility and dynamics from nanofiber mechanics, and allows the peptides to move, or “slide”, along the PA nanostructures. A peptide being able to move or slide along the PA nanostructure is referred to herein as “mobile”. This is visualized schematically in FIG. 1C. Importantly, it enables the peptides to be mobile enough to detach from the fibers and impart their bioactivity intracellularly.

The slider system that incorporates TAT-conjugated ISP and DSLK was compared to analogues in which the TAT moiety is replaced with a shorter, less charged sequence (R4). Interestingly, regardless of the bioactive epitope, the TAT-containing sliders exhibited significantly lower mobility compared to their R4 and control analogues and induced fiber bundling, indicating a tunable dynamic structure. The effect of ISP and DSLK peptides and sliders on primary mouse cortical neurons was evaluated. Initial assays confirmed the ability of the TATISP and TATDSLK peptides to overcome CSPG-induced growth inhibition. Morphometric analysis revealed that the bioactivity of the peptides was maintained when bound to PA nanofibers within the slider system. These results were corroborated with a series of in vitro models of SCI using both healthy and injured neurons in the presence of CSPGs. The TATISP and TATDSLK sliders exhibited far greater and prolonged bioactivity compared to the soluble peptides, resulting in neurite regrowth in an inhibitory environment.

### Slider Synthesis

Slider fabrication involves pre-forming negatively charged PA fibers using backbone molecule C₁₆V₂A₂ E2 (E2). The E2 molecules are synthesized using standard Fmoc-based solid-phase peptide synthesis (SPPS). The lyophilized powder is first solubilized in phosphate-buffered saline (PBS) and annealed to form strong fibers approximately 10 nm in diameter. To characterize how the binding and mobility of ISP-based sliders are impacted by the positively charged region attached to the bioactive peptides, both the TAT and R4 sequences were tested, along with control peptides containing no positive charges (FIG. 1). Small amounts of peptides are added incrementally to the pre-formed E2 fibers, followed by immediate vortex mixing to establish a homogenous mixture.

### Structure of ISP-Based Sliders

To determine whether the supramolecular structure of the PA fibers was maintained with peptide addition, small-angle x-ray scattering (SAXS), wide-angle x-ray scattering (WAXS), and circular dichroism (CD) were performed. All SAXS and WAXS samples were made by annealing E2 PA fibers at 5 mM in PBS and adding respective peptides at 0.25 mol %. immediately followed by 20 s of vortex mixing. CD samples were prepared by further dilution to 50 μM in MiliQ water. A flow-cell setup was used for all SAXS and WAXS measurements.

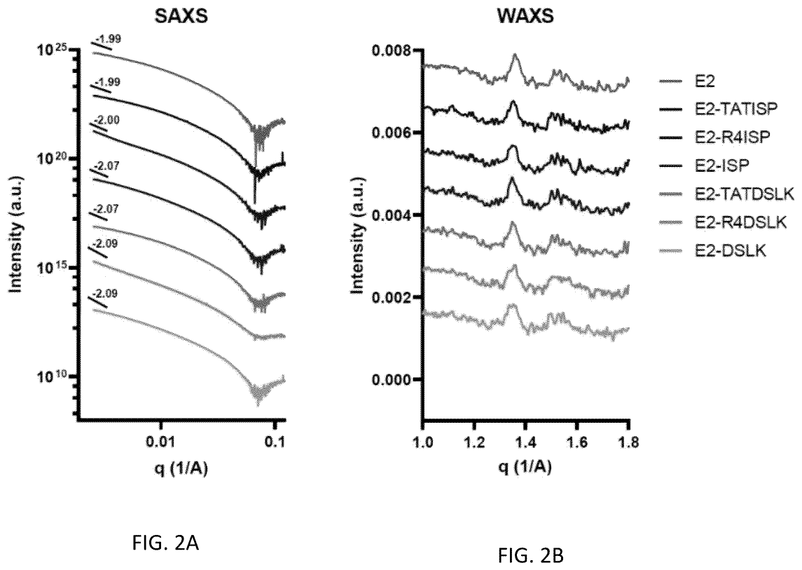

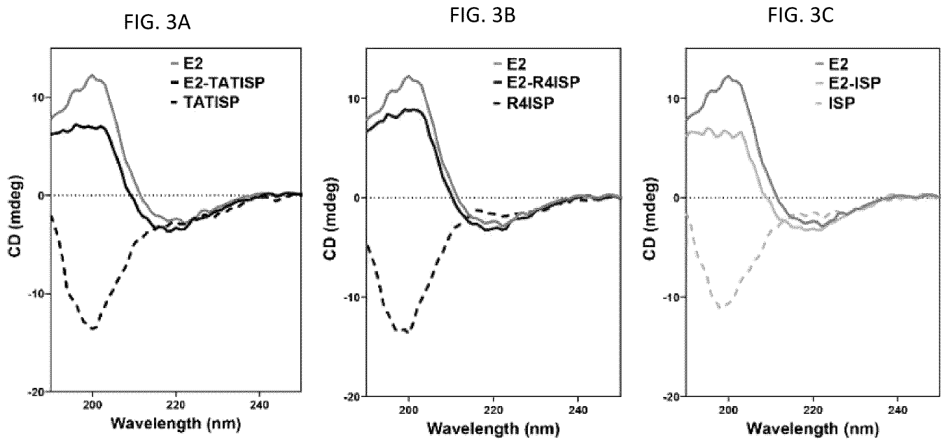

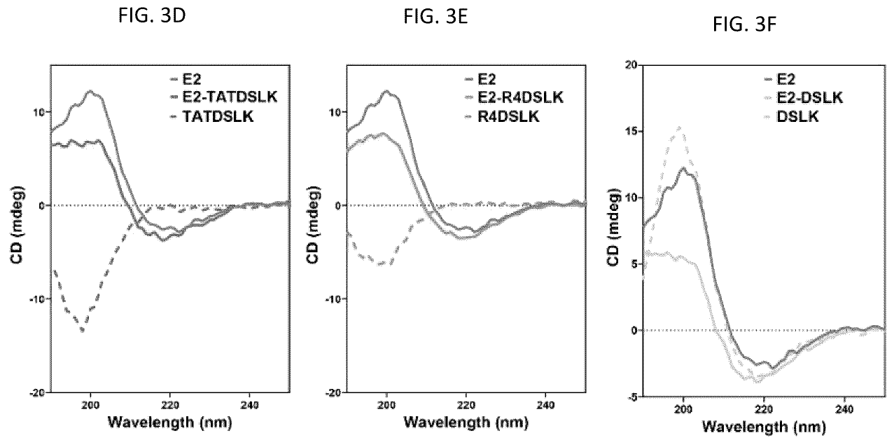

FIG. 2 shows the SAXS and WAXS profiles. All sliders, regardless of the positively charged sequence, provided SAXS slopes similar to the unbound E2 PAs in the Guinier region. These slopes are around −2, indicating ribbon-like morphologies. This suggests that addition of 0.25 mol % peptides does not disrupt the ribbon-like morphology of E2 nanofibers. Similarly, the intensity of the β-sheet peak at 1.35 Å⁻¹ in the WAXS region is similar among all slider compositions, suggesting that the β-sheet secondary structure is also maintained upon peptide binding. This is corroborated with CD, as shown in FIG. 3. All soluble peptides, apart from DSLK, demonstrate random coil structures when unbound to PA fibers. However, when added to E2, they contain a negative peak around 220 nm, indicative of β-sheet secondary structure. Interestingly, DSLK alone also shows β-sheet conformation, indicating that this peptide can form similar secondary structures to PA fibers.

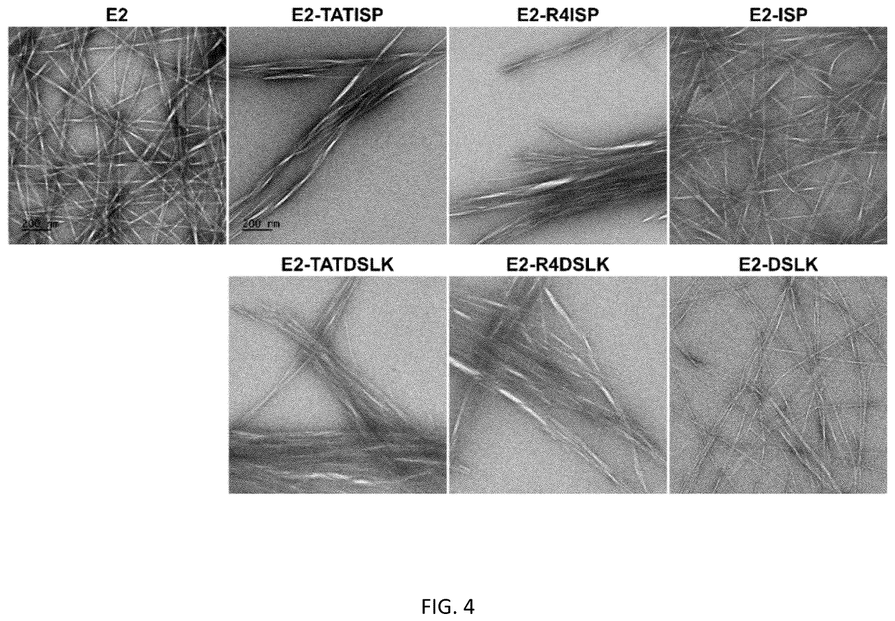

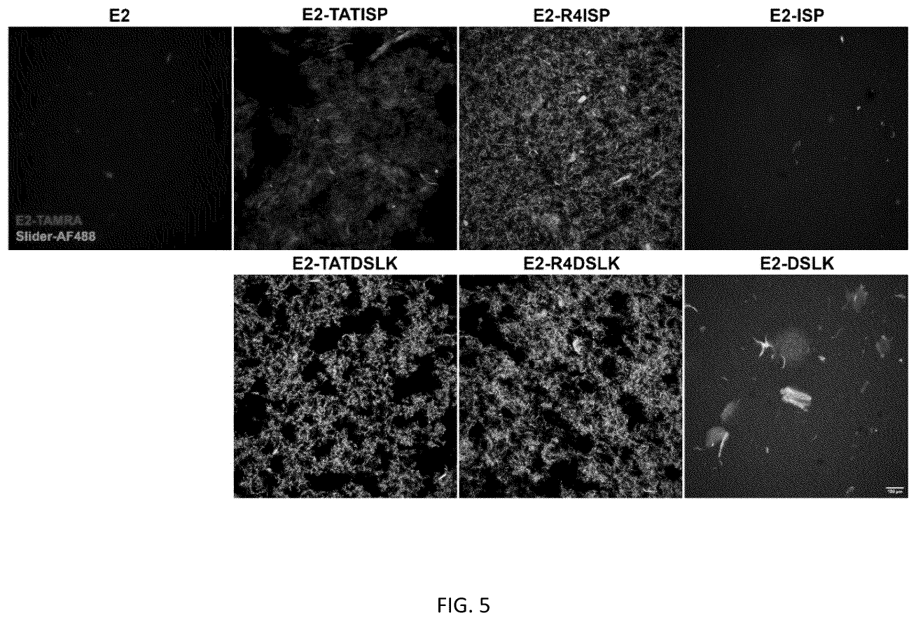

Transmission electron microscopy (TEM) was performed to validate the preservation of fiber morphology. All TEM samples were prepared by making E2 PA fibers at 2 mM in PBS and annealing at 80° C. for 1 hour. Sliders were made by diluting the annealed E2 fibers to 1 mM in PBS and adding 6.25 mol % of the respective peptides, immediately followed by 20 s of vortex mixing. Samples were templated onto TEM grids and imaged using conventional TEM. As seen in FIG. 4, all compositions showed fibrous structures. However, PA fiber bundling is induced upon the addition of peptides containing a series of positively charged amino acids. Both ISP and DSLK peptides with either TAT or R4 caused similar levels of bundling, whereas ISP and DSLK peptides without positive charges maintained individual fiber morphology of E2 PAS.

### Binding and Mobility of ISP-Based Sliders

To confirm the binding of the peptides to E2 fibers, various fluorescent techniques were used, including scanning confocal microscopy, fluorescence recovery after photobleaching (FRAP), and Förster resonance energy transfer (FRET). Fluorescently labeled fibers were made by annealing 2 mM E2 with 0.1 mol % E2 conjugated to 5-Carboxytetramethylrhodamine (TAMRA) on the C-terminus. Unlabeled peptides and 0.1 mol % peptides conjugated with AlexaFluor-488 (AF-488) were then added, immediately followed by 20 s vortex mixing.

FIG. 5 shows high degrees of colocalization between the fibers and peptides, as observed by the overlap of fluorescence. Additionally, homogenous fiber morphology is observable for E2 alone, whereas bundling of fibers is noticeable for the sliders containing TAT and R4 sequences, further corroborating the previous TEM results. While no bundling was observed with the sliders without a positively charged region, there was observable overlap of the peptides and PA fibers. One possible explanation for this is the presence of positively charged residues found in the bioactive regions, which may also interact with the negative fibers.

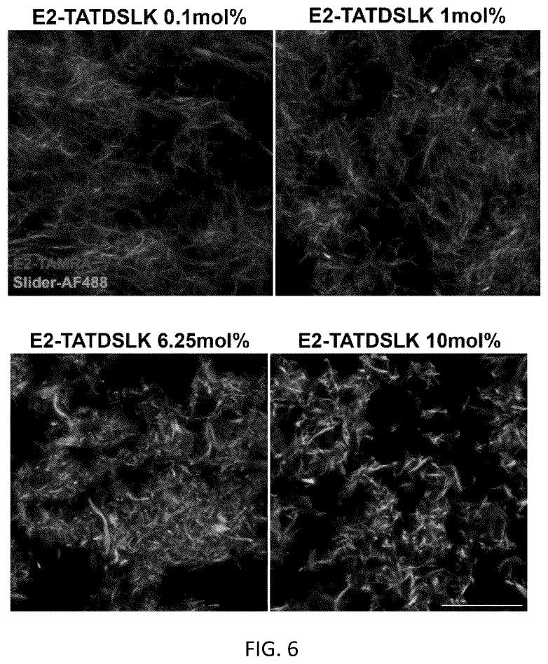

To further investigate how bundling is impacted by peptide addition, sliders were made with varying concentrations of TATDSLK and imaged under confocal microscopy (FIG. 6). Interestingly, a higher degree of bundling is seen as the peptide concentration is increased. Without wishing to be bound by theory, it is hypothesized that this bundling is caused by the long series of positive charges electrostatically interacting with multiple fibers, thus acting as interfiber linkers. Another potential cause is the partial neutralization of the negative charge on the fiber surface upon peptide binding, thus further reducing interfiber distance.

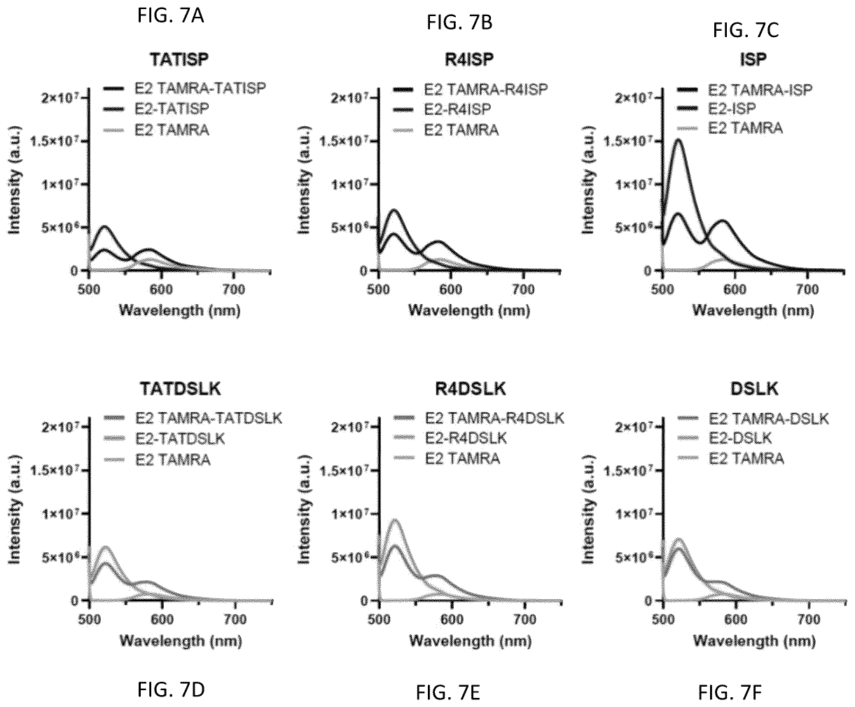

As a confirmation of binding between the peptide and fiber, FRET was performed using AF-488 conjugated peptides as the donor and TAMRA-conjugated E2 PA fibers as the acceptor. The proximity of the donor-acceptor dye pair, caused by binding of the peptide to the nanofiber, induced FRET. This was observed as a quenching of the emission band of AF-488 and heightening of the emission band of TAMRA upon stimulation with 468 nm (FIG. 7).

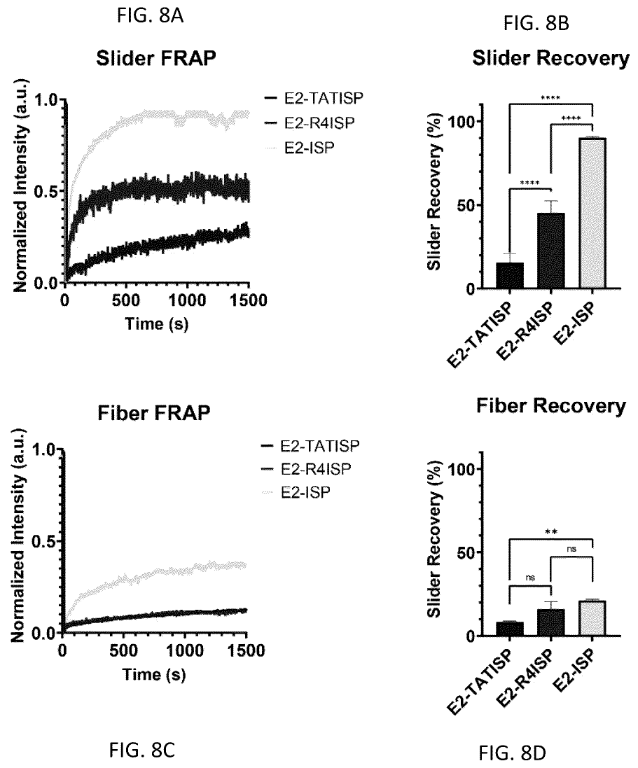

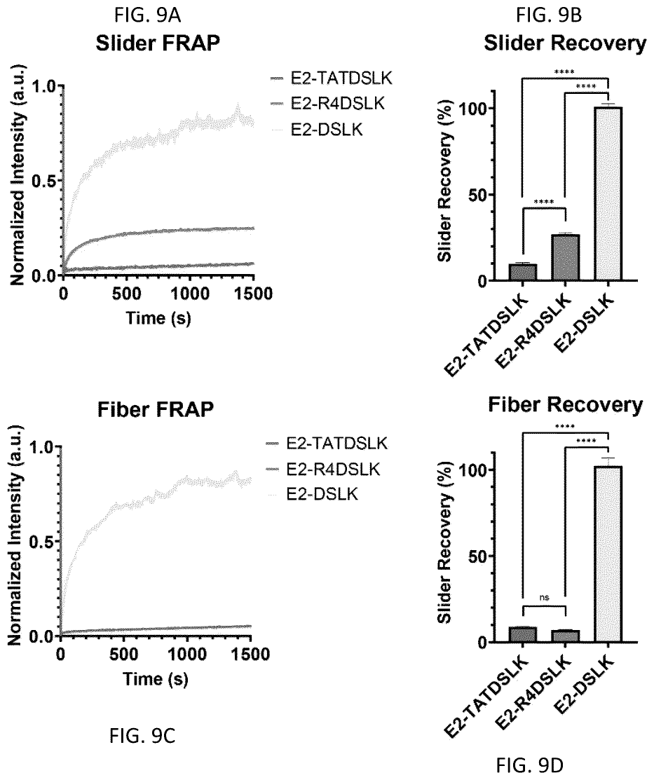

To determine how slider mobility is impacted by the number of positive residues attached to the bioactive epitope, FRAP was performed on sliders with 6.25 mol % peptide. Prepared samples were deposited onto a glass slide within a thin circular spacer, and carefully covered with a glass coverslip to avoid evaporation during imaging. Three ROIs were drawn within a field of view, and 15 seconds of pre-bleaching images were acquired. The 484 and 561 lasers were used to photobleach the areas for one second, followed by image acquisition every second for one hour. FIG. 8 and FIG. 9 show fluorescence recovery of the peptides and E2 fibers within the sliders for ISP and DSLK, respectively. Sliders containing the TAT sequence had significantly lower recovery compared to their R4 or control counterparts, indicating a strong interaction with the nanofibers. Those with an R4 sequence demonstrated an intermediate level of mobility, consistent with the reduction in positive residues. Interestingly, the addition of the sliders lowered the recovery of the E2 fibers, suggesting a reduction in mobility of fiber molecules. We hypothesize that the fibers are further stabilized by peptide addition due to the positively charged arginines and/or lysines reducing the intrafiber repulsive forces of adjacent glutamic acids.

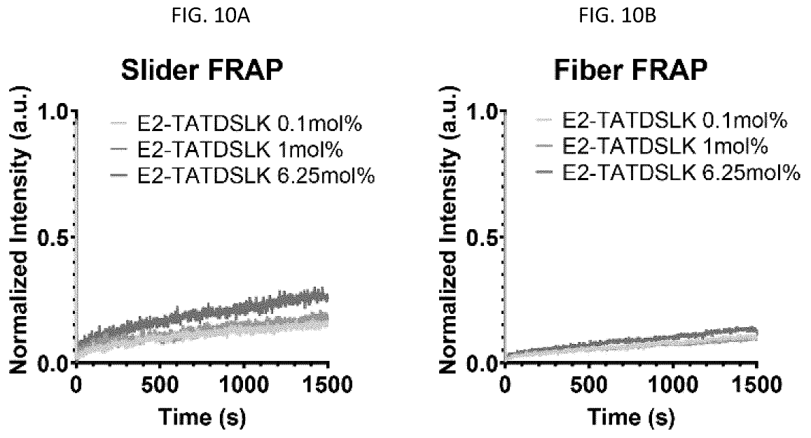

To investigate whether the slider mobility is influenced by bundling, FRAP was tested on different concentrations of TATDSLK. FIG. 10 shows similar peptide recovery regardless of TATDSLK concentration, which as discussed above is linked to degree of bundling. It can therefore be concluded that the low mobility of the TAT sliders is a function of the number of positive charges within the TAT sequence, rather than the fiber morphology.

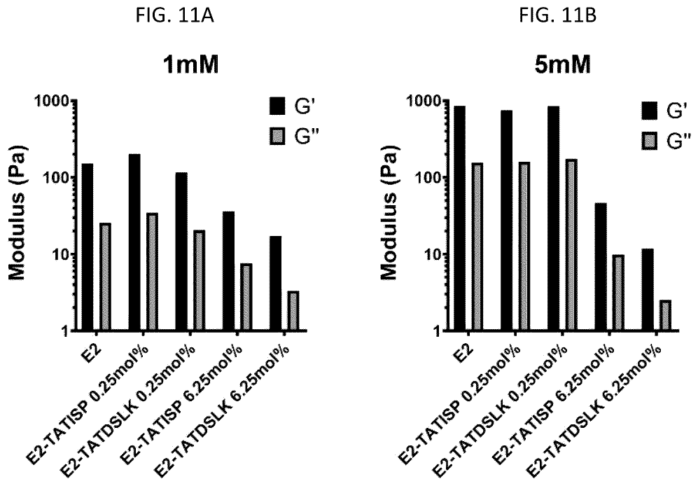

One of the benefits of PA materials is the tunability of the mechanical properties depending on the target application. To evaluate how the gelation of sliders is impacted by peptide addition, rheology was performed at a low (1 mM) and high (5 mM) concentration of E2 PAs with either a low (0.25 mol %) or high (6.25 mol %) concentration of TATISP and TATDSLK. To better mimic in vitro and in vivo environments, measurements were taken at 37° C. and in the presence of physiologically relevant CaCl₂ concentration. At low PA concentration, there is little gelation observed for any of the conditions (FIG. 11). However, when the E2 fibers are at 5 mM, the moduli are indicative of hydrogels, and this is maintained with the addition of 0.25 mol % peptides. However, higher peptide concentrations reduced these values, demonstrating an impact on their mechanical properties. It is hypothesized that the bundling induced by higher concentrations of TAT-conjugated peptides interrupts the crosslinking between PA nanofibers. This idea can be applied to alter the gelling state of sliders, depending on the target tissue and application. For example, if the material is to diffuse throughout the tissue surrounding the injury cavity in SCI, a material with low gelation properties may be ideal.

### Discussion

The work herein demonstrates that the slider system can be modified to use PA nanofibers as a platform for ISP-based peptide delivery. The ISP peptide was modified to contain only the last 12 residues (a truncate referred to as “DSLK”). ISP and DSLK peptides containing varying degrees of positively charged moieties were developed, synthesized, and characterized as a slider system. In the initial design, peptides were conjugated to a TAT sequence that allows cell penetration for intracellular signaling. To study how this highly positive sequence impacts the electrostatic binding to negatively charged PA nanofibers, TAT was replaced with a sequence of four arginines (R4) that reduced the net positive charges. These peptides were added to E2 PA nanofibers as a slider system and compared to sliders with peptides containing no positive region.

SAXS, WAXS, CD, and TEM confirmed that the structure and β-sheet character of the preformed E2 PA nanofibers were relatively maintained with the addition of the peptides. The conservation of fiber morphology is beneficial for regenerative purposes, as it greatly mimics the structure of the ECM. TEM images revealed PA fiber bundling in the sliders containing TAT and R4 sequences. This was further probed with fluorescent imaging, confirming a bundling effect with positively charged sliders but not the control sliders. The bundling is dependent on the peptide concentration, with lower concentrations maintaining the individual fiber nature.

These results also indicated binding of the TAT and R4 peptides to the PA nanofibers, which FRET experiments further validated. FRAP was used to study the mobility of the slider systems and revealed an interesting trend. TAT-based sliders had low fluorescence recovery, suggesting low mobility, whereas R4-based sliders had significantly more mobility. The control sliders showed full fluorescence recovery, indicating these peptides have little association with the E2 fibers. Importantly, the low mobility of TAT sliders is not concentration or bundling dependent. Upon studying the fiber recovery, sliders with both TAT and R4 were shown to greatly reduce fiber mobility. Binding of strongly associating peptides may have a stabilizing effect, which could be beneficial for long-term applications.

### Example 2

During maturation, neurons extend long axons to connect with dendrites of other neurons to conduct electrical signals. Upon damage to the axon, the end distal of the injury site degenerates completely. However, the proximal end is still attached to the cell body and is capable of regenerating following a slight retraction. Unfortunately, inhibitory molecules surrounding the SCI site, such as CPSGs, prevent this regrowth. This example assesses the ability of the ISP-based slider system to overcome the inhibitory effects of CSPGs on neuron growth, survival, and maturation.

### Morphometric Analysis

At the growing end of the axon is a growth cone, which senses and responds to environmental cues for growth. The growth cone contains high numbers of filopodia and lamellipodia, and the structure is heavily reliant on actin filament nucleation, polymerization, and turnover. Extrinsic inhibitors of axon regeneration, such as CSPGs, cause the growth cone to form a bulb-like structure, known as a dystrophic growth cone. While the dystrophic growth cone is dynamic and holds the capability of regenerating, it ultimately stalls forward motion and prevents the neurite from extending.

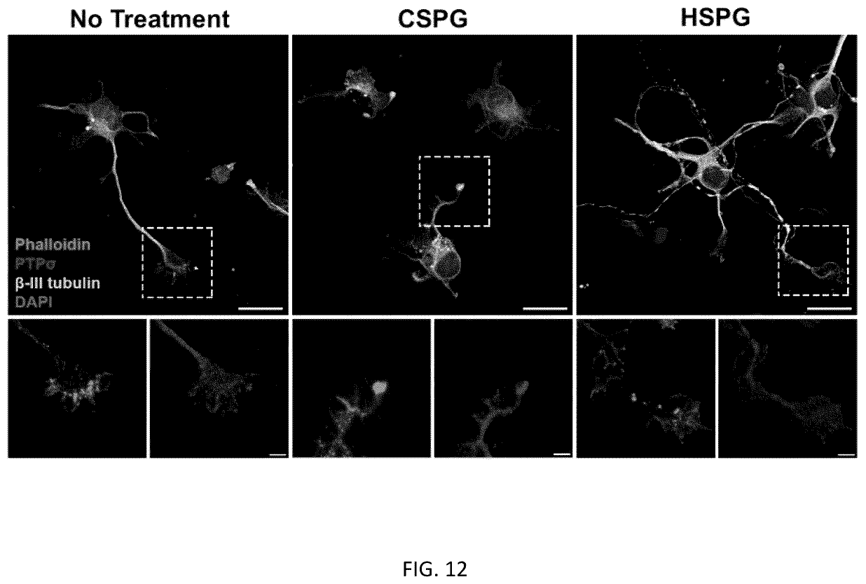

To observe this phenomenon in vitro, primary mouse cortical neurons were cultured with the addition of either CSPG, or its opposing molecule, HSPG, in media. FIG. 12 shows this effect clearly, where neurons grown on a poly-D-lysine (PDL) substrate exhibit growth cones with a hand-like structure and extending filopodia. However, when even a low concentration of CSPG is introduced, the growth cone forms a bulb-like structure, and the filopodia are no longer observed. The addition of HSPG at the same concentration leads to a large growth cone similar to the PDL control, supporting their ability to induce opposite effects compared to CSPGs. Another characteristic of CSPG-induced inhibition is the localization of PTPσ to the dystrophic growth cone, as shown in FIG. 12.

To assess how TATISP and TATDSLK impact neuron growth with and without CSPGs, they were used to treat primary mouse cortical neurons grown on either PDL or a CSPG substrate. A coating of 10 μg/mL laminin and 1 μg/mL CSPG, was used to inhibit the survival and growth of neurons. After letting the cells adhere to the surface for four hours, TATISP and TATDSLK, as well as their respective sliders, E2-TATISP and E2-TATDSLK, were added in the media. For all in vitro experiments, unless otherwise noted, the final peptide concentration remained consistent at 2.5 μM. Sliders were prepared at 1 mM E2 PA with 6.25 mol % peptide and were added at 40 μM final PA concentration. Non-bioactive E2 PA was used as a control, as well as no treatment. After 48 hours of treatment, the cells were fixed and stained with the neuronal marker, β-tubulin, and with an actin filament stain, phalloidin.

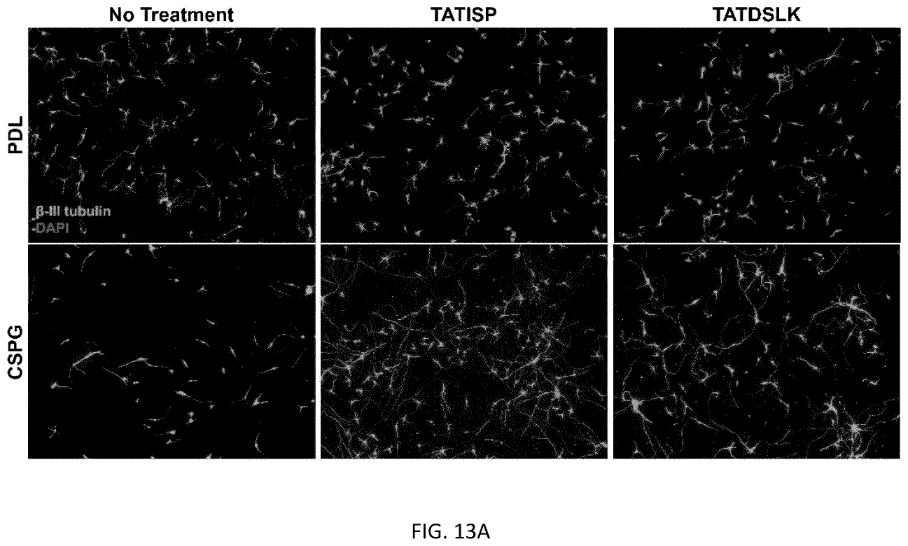

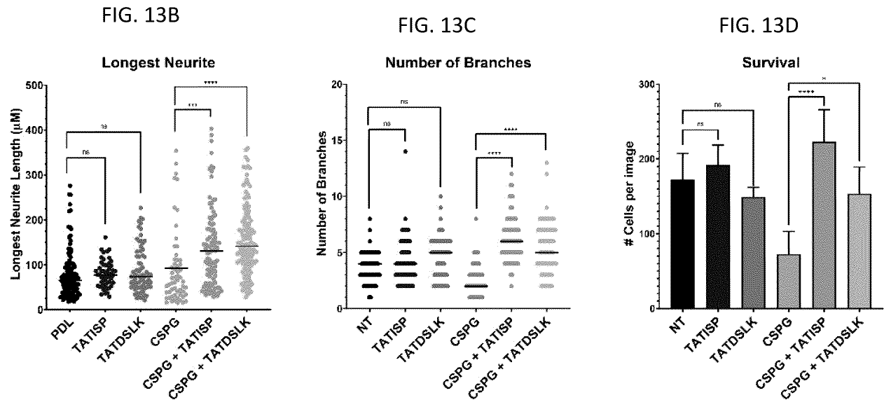

FIG. 13 shows the effect of the soluble TATISP and TATDSLK peptides on neurons grown on either PDL or CSPG. Neurons grown on PDL and treated with peptides did not show a morphological difference from the no treatment control. Interestingly, neurons on the CSPG coating and treated with TAT-based peptides exhibited significantly longer neurites compared to those seeded on the PDL control. Additionally, the number of surviving neurons was increased compared to the CSPG control. This demonstrates the ability of TATISP and TATDSLK to counteract the inhibitory effects of a CSPG substrate after 48 hours of treatment.

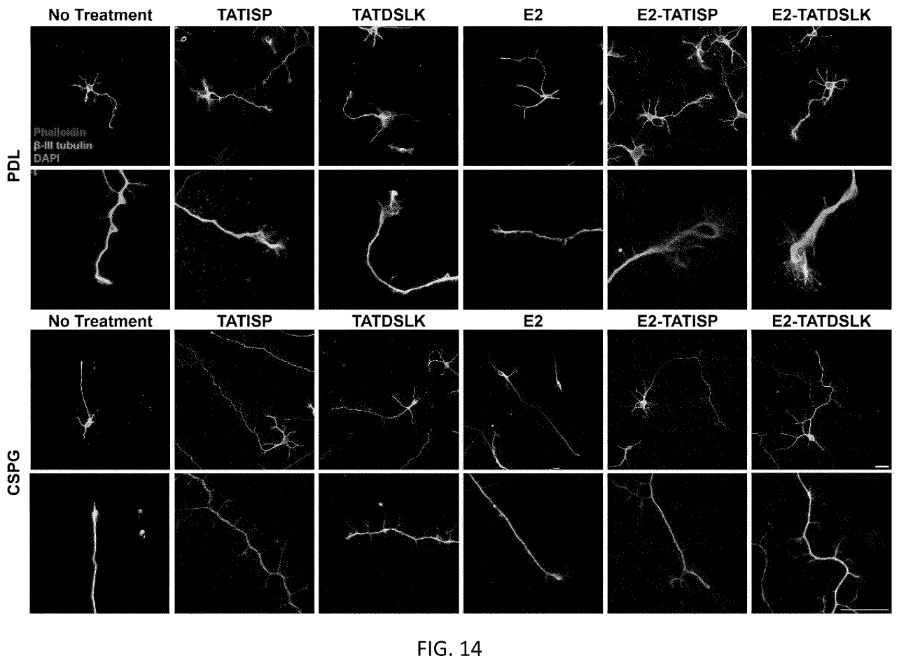

Upon closer examination, there seemed to be an impact on neuron morphology for both the peptides and sliders, as shown in FIG. 14. While the length of the primary neurite did not change within the groups seeded on PDL, the growth cone area was larger in those treated with TATISP and TATDSLK soluble peptides, and even more-so with the sliders. Interestingly, the surviving neurons on the CSPG coating displayed a clear morphological difference regarding the number of branches. Untreated cells in this group had few primary branches, and even fewer spine structures protruding from those neurites. When treated with TATISP or TATDSLK peptides, the number of primary branches and filopodia structures increased. Similar observations were made with the TATISP and TATDSLK sliders, indicating the bioactivity of the peptides was maintained when bound to E2 PA nanostructures. Meanwhile, the nonbioactive E2 PA condition showed similar morphology to the no treatment control, suggesting the role of the peptides in CSPG-mediated inhibition.

### CSPG Relief

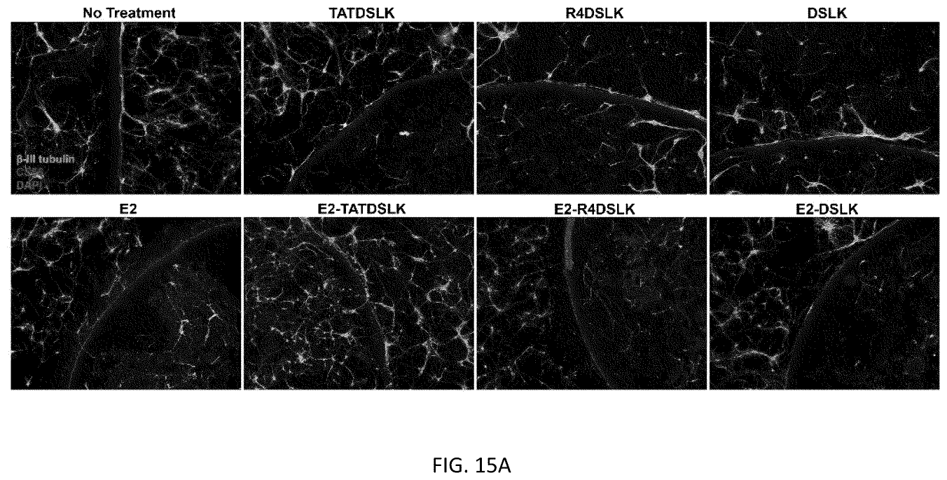

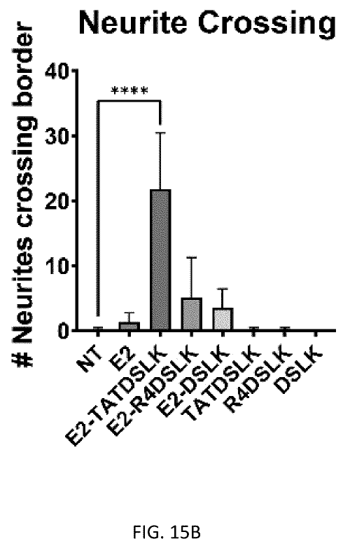

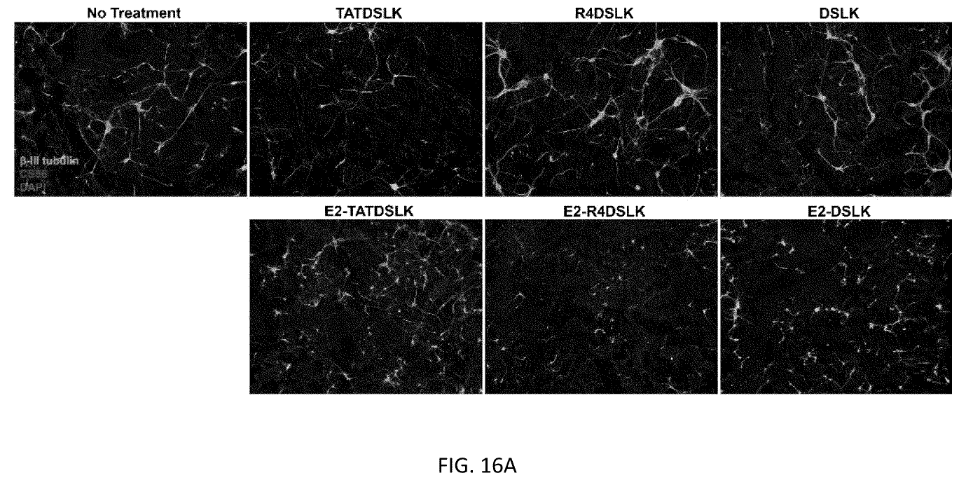

To characterize the sliders' biological effect in a more accurate mimic of the CSPG barrier surrounding the injury lesion, a spot assay was performed. A solution of laminin and CSPG was dropped onto PDL-coated glass coverslips, resulting in a gradient of growth-inhibitory CSPGs that counteracts a reverse gradient of growth-promoting laminin. The composition of this assay portrays the dense CSPG barrier mixed with ECM proteins, such as laminin, that the injured neurons approach in chronic SCI. Primary mouse cortical neurons are then seeded on top of the spots at a high density and allowed to adhere for one day. They were treated with DSLK-based sliders or soluble peptides and grown for five days. As shown in FIG. 15, untreated neurons were not able to cross over the CSPG barrier, visualized through immunostaining with CS56 in red. The one-time treatment with the soluble DSLK peptides did not result in significant neurite crossing. Interestingly, neurons treated with sliders containing either the R4-conjugated DSLK or the control DSLK peptide (no positive charges) resulted in some, but not many, neurite crossings. However, the slider containing TATDSLK significantly increased the number of neurites crossing over the CSPG border, indicating the requirement of the TAT sequence for bioactivity.

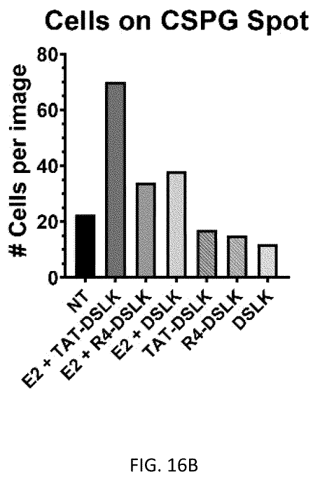

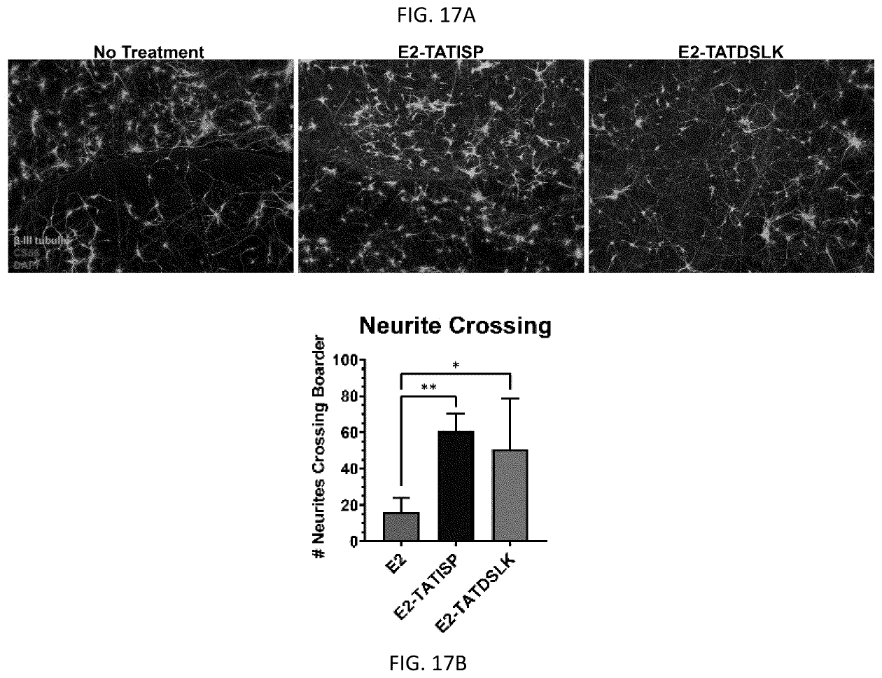

Similar observations are made when comparing the number of cells surviving on the CSPG spot, where only the slider with TATDSLK showed significantly higher cell survival compared to the no treatment condition (FIG. 16). These results suggest the sustained delivery of TATDSLK on PA nanostructures can greatly improve bioactivity compared to the TATISP soluble peptide alone. Because the sliders without the TAT sequence were unable to promote neurite crossing and survival, the following experiments focused only on the TAT versions of the sliders and peptides. When comparing TATISP to TATDSLK sliders, TATISP allowed for higher number of neurite crossings over the CSPG spot, yet both significantly improved crossings compared to E2 PA control (FIG. 17).

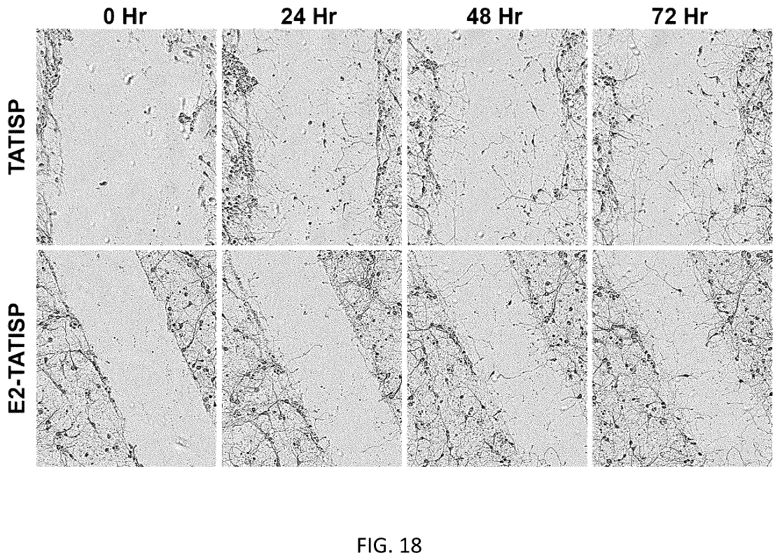

To validate these findings, another injury model was prepared in vitro. Here, primary mouse cortical neurons were grown into a confluent 2D layer for seven days before being mechanically cut by scraping the surface with a pipette tip. Immediately following the injury, CSPGs were added with and without TAT-based peptide or slider. To observe the regrowth rate, images were acquired on an IncuCyte imaging system every six hours for 72 hours. As seen in FIG. 18, the soluble TATISP peptide induced neurite growth within the first day but slowed down the following two days. However, growth seen in the E2-TATISP slider condition began after the first day and continued until day three, indicating prolonged bioactivity compared to the soluble peptide version.

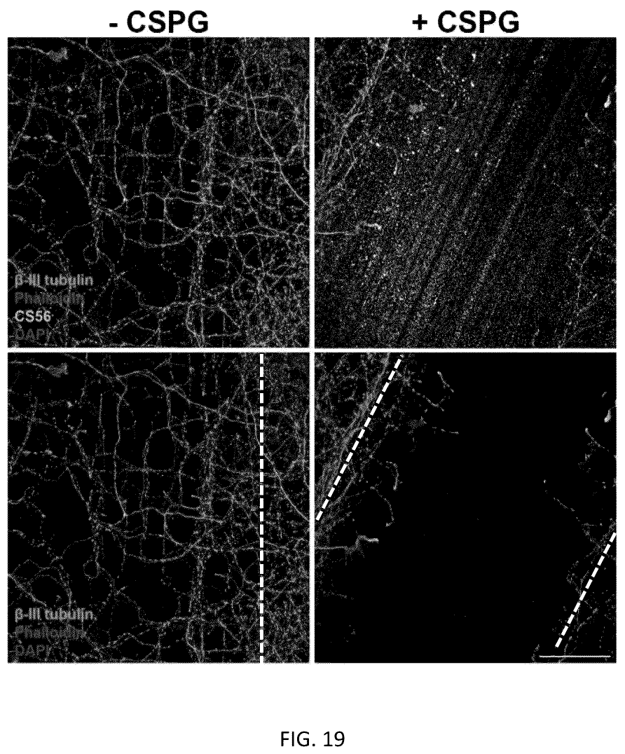

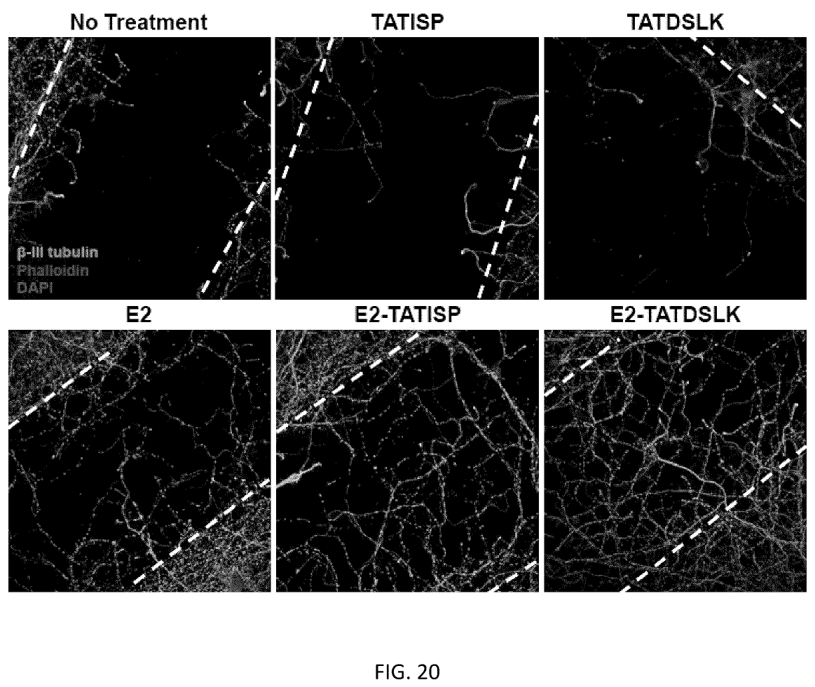

Further visualization was accomplished using immunocytochemistry, where CSPG, actin, and β-tubulin were stained. CSPG was found to be localized to the scratch area, as shown in gray in FIG. 19. The condition where no CSPG was added did not have any CSPG present within the scratch, and the neurites were seen to grow well within the injured area compared to the CSPG condition. This further demonstrates the inhibitory nature of CSPG accumulation following neuron damage, as seen in chronic SCI. FIG. 20 shows the impact of neurite crossing in the presence of CSPG after mechanical damage when treated with TAT-based peptides or sliders. Upon treatment with the soluble TATISP and TATDSLK peptides, there were neurites that began extending into the scratch area but did not completely cross over. However, neurons treated with both the TATISP and TATDSLK sliders showed significantly more neurites completely crossing the scratch area. These results demonstrate that the combination of TATISP or TATDSLK with E2 as a slider system promotes neuron regrowth following injury, even in the presence of CSPGs.

### Intracellular Inhibition of PTP and Synaptogenesis

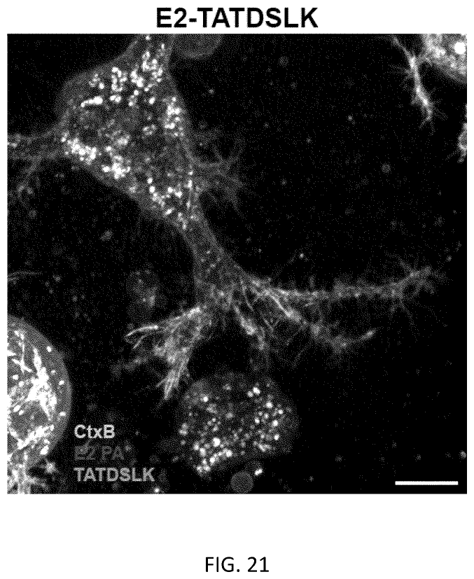

The above results demonstrate the bioactivity of the ISP-and DSLK-based sliders. Their mechanism of action was next evaluated. The association of the sliders with neurites can be observed after 72 hours (FIG. 21). The uptake capacity of the bioactive peptides when bound to PA fibers was evaluated. It was hypothesized that the TAT-PA interaction is strong enough to prolong the peptide delivery, yet weak enough that the peptides can enter the cell to interact with PTP receptors.

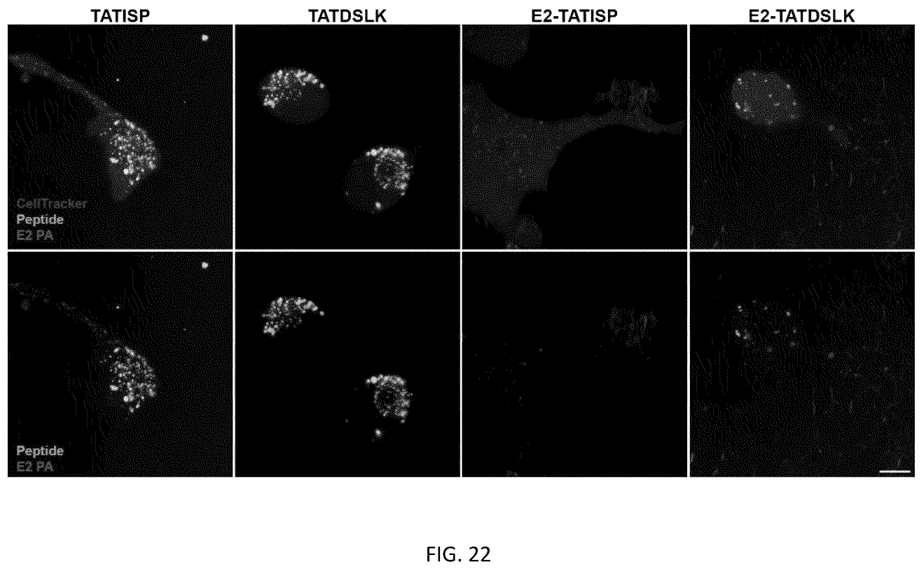

To investigate the uptake capacity of the TAT-based materials, live fluorescent imaging was used on Neuro-2a (N2a) cells treated with either AF-488 conjugated TATISP or TATDSLK, or with sliders containing AF-488 -TATISP or TATDSLK and E2-TAMRA PAS. To visualize the cytoplasm, CellTracker blue was used. FIG. 22 shows the uptake TAT-based peptide and slider after 72 hours. It can be observed that high amounts of peptide are internalized, whereas slider uptake is minimal.

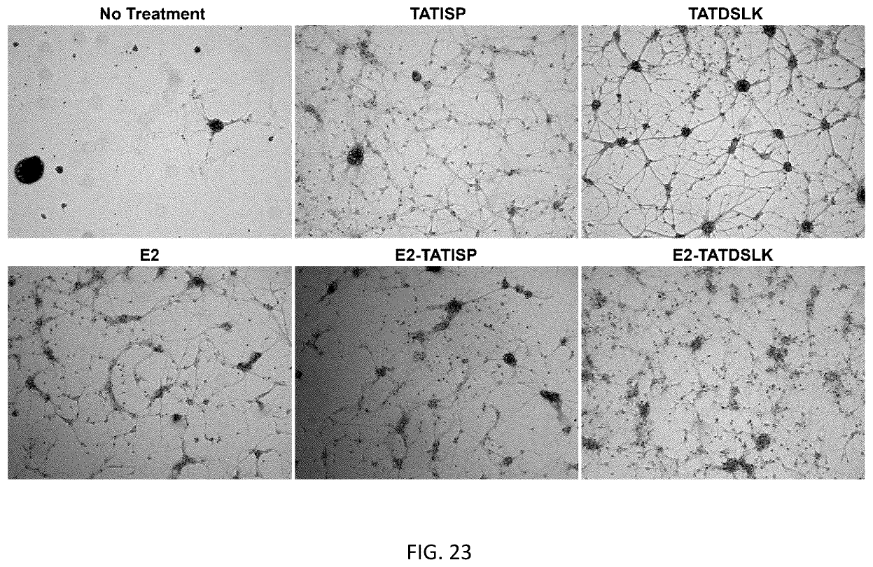

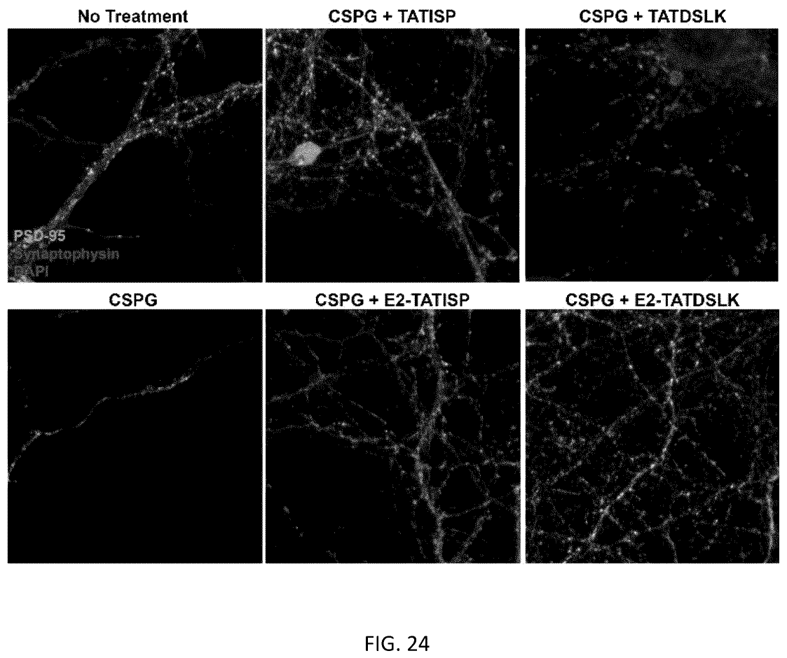

Apart from axon extension, the re-establishment of synaptic relays is a critical factor in successfully restoring function following CNS injury. To study the long-term impact of TAT-based peptides and sliders, primary cortical neurons were seeded on PDL-coated coverslips and treated with 3 μg/mL CSPG with or without TATISP and TATDSLK peptides or sliders in media. A half-medium change was performed every 3-4 days, and the treatments were replenished during each change. After 14 days, there was apparent cell death and clustering in the CSPG condition (FIG. 23), indicating unhealthy cultures. While cell survival was enhanced with TATISP and TATDSLK peptides, there were still high numbers of cell clusters. Fewer clustering was seen with the TATDSLK slider, indicating healthier cultures. These cultures were then stained with synaptic markers, PSD-95 and synaptophysin (FIG. 24). There was an observable difference in the number of synapses with the TATDSLK slider condition compared to all other conditions. This may indicate the ability of this particular material to promote synaptogenesis in the presence of CSPGs, a property that would greatly benefit regeneration following chronic SCI.

### Discussion

Herein it was investigated whether ISP-based sliders could overcome CSPG-induced inhibition in primary cortical neurons. First, the bioactivity of TATDSLK peptide was tested using neurons growing on a CSPG substrate. While there were no obvious differences in cellular behavior and morphology on a PDL substrate, after 48 hours TATDSLK demonstrated a similar recovery of growth-inhibited neurons compared to TATISP on a CSPG substrate. This suggests that PTPσ may be inactivated with both peptides when CSPG is present.

The DSLK-based slider system was then tested on a CSPG spot assay to assess prolonged activity in an SCI-mimetic environment. Neither the TAT, R4, or control soluble peptides promoted neurite crossing over the inhibitory barrier. However, the TATDSLK slider greatly enhanced the number of neurite crossings and survival on the CSPG spot, indicating its prolonged bioactivity compared to the soluble peptide analogue. The TATISP slider showed similar results, whereas the nonbioactive E2 PA control showed minimal crossing. These results confirm the necessity of combining TATISP or TATDSLK with nanostructures for a long-term effect. This trend was consistent with a scratch assay, in which TATISP and TATDSLK sliders prompted neurite regrowth into a damaged area, even in the presence of CSPGs.

The uptake and prolonged effect on neuronal connectivity was further investigated. Although there was association of the sliders with N2a cells, after 72 hours there was little observable internalization compared to the soluble TAT-based peptides. The strong interaction between the TAT sequence and the negatively charged PAs may prevent uptake within this timeframe, or the amount of internalized peptide may be too low to observe. However, prolonged exposure to the TATDSLK slider shows a clear effect on enhancing synaptic markers. This suggests that over time, the slider has enhanced bioactivity compared to the soluble peptide.

### Example 3

### Materials and Methods

### Peptide and PA Synthesis

PA molecules were synthesized by fluorenylmethoxycarbonyl (Fmoc) solid-phase peptide synthesis (SPPS) on rink amide resin. PA molecules and peptides were purified using reverse-phase high-performance liquid chromatography (HPLC). PA molecules and peptides labeled with either 5-Carboxytetramethylrhodamine (TAMRA) or Alexa-Fluor 488 (AF-488 ) were synthesized with an added lysine on the C-terminus of the sequences. PA molecules and peptides were lyophilized into a dry powder and stored at −30° C.

### Slider Preparation

Lyophilized PA powder was dissolved in PBS at either 2 mM or 10 mM and pH adjusted to ˜7.4 by adding 1M NaOH. Samples were sonicated for 30 minutes, then annealed in an 80° C. water bath for 1 hour, and slowly cooled overnight to room temperature. For fluorescently labeled PA solutions, E2 dissolved in PBS was mixed with 0.1 mol % E2-Tamra in PBS, pH adjusted to ˜7.4 with 1M NaOH, sonicated, and annealed. PA solutions were further diluted in PBS and vortex mixed for ˜20 seconds. Lyophilized peptides were dissolved in water at 1 mM. Slider solutions were prepared by adding peptides to the E2 solution, 5 μL at a time, to the wall of the Eppendorf tube. Solutions were immediately vortex mixed ˜20 seconds after each addition.

### Small- and Wide-Angle X-Ray Scattering

Slider samples were prepared as previously described, with 5 mM E2 concentrations and peptides added at 0.25 mol %. X-ray scattering experiments were performed at Argonne National Lab at Sector 5 IDD using a flow-cell. All data was background subtracted prior to analysis.

### Circular Dichroism

Slider samples were prepared as previously described at 1 mM E2 concentration with peptides added at 1 mol %. Samples were diluted to 50 μM in milliQ water. 190 μL samples were loaded into a quartz cell of 1 mm optical path length. CD spectra were recorded. Three measurements were taken per sample and normalized.

### Transmission Electron Microscopy

Samples were prepared with 1 mM E2 final concentration and 6.25 mol % slider. To prepare the negatively stained TEM samples, 7 μL of PA solution was dropped onto plasma cleaned TEM grids for 15 seconds, then wicked away, and washed twice with water. All PA samples were negatively stained with 2 wt % uranyl acetate (UA) for 25 seconds, then wicked away. Samples were dried at room temperature prior to storage and then imaged.

### Confocal Laser Scanning Microscopy

Fluorescence microscopy of slider samples was performed on a Nikon-A1R confocal microscope. Fluorescent samples were made at 1 mM E2 with 0.1 mol % E2-Tamra, 0.1 mol % Alexa-Flour488-conjugated peptides, and 6.14 mol % unlabeled peptide. 10 μL slider samples were deposited into a spacer on a clean microscope slide and covered with a glass coverslip. Samples equilibrated for ˜10 minutes upside down, and images were captured with a 20× air objective.

### Förster Energy Resonance Transfer

Förster energy resonance transfer (FRET) spectroscopy was performed in a Cytation 3 plate reader. Slider samples were prepared using 0.1 mol % of the fluorescently labeled peptides and PAs. The emission was measured in a 384-well plate with stimulation at 468 nm. Control samples consisting of unlabeled E2 PAs with AF-488 peptides were used to account for the nanostructures.

### Fluorescence Recovery after Photobleaching

Fluorescence recovery after photobleaching (FRAP) was performed on a Nikon-A1R confocal microscope with a Plan Apo 10× objective. A series of images was captured every second for 15 seconds to establish a baseline fluorescence within three circular regions of interest (ROI) (15 μm diameter). The ROIs were then photobleached with the 484 nm and 561 nm lasers at 100% and 30% power, respectively, for 1 second, and images were then captured every second for 1 hour. Analysis was performed in ImageJ using the Stower's Institute plugin. Samples were bleach-corrected with an exponential fit, and fluorescence recovery over time was normalized for each ROI and averaged.

### Rheology

Slider samples were prepared as described above with either 0.25 mol % or 6.25 mol % TATISP and TATDSLK. The stage of an MCR302 Rheometer was set to 37° C. 150 μL of slider solution was placed onto the stage, and 30 μL of 25 mM CaCl₂ solution was placed onto the underside of a 25 mm cone plate above the material in evenly spaced drops. After lowering the plate, a humidity collar was placed to prevent sample evaporation. The samples were equilibrated for 30 minutes with a constant angular frequency of 10 rad/s and 0.1% strain. The storage and loss modulus (G′ and G″) were recorded after a plateau developed.

### E16 Primary Mouse Cortical Neuron Dissection and Culture

Primary neurons were obtained from embryonic mouse brains. A time pregnant CD1 mouse was sacrificed by cervical dislocation and the embryos were extracted on embryonic day 16 (E16). Cerebral cortices were dissected, and meninges removed. Cortices were placed in a solution of Hank's Balanced Salt Solution (HBSS) with 1% pen-strep, followed by digestion in 0.25% Trypsin/EDTA with DNAase for 10 minutes at 37° C. Cortices were mechanically dissociated and let to settle. The supernatant was then transferred and centrifuged at 1000 rpm for 5 minutes, and the pellet was resuspended in CO₂-equilibrated Neurobasal medium supplemented with 10% normal horse serum (NHS), 1% pen-strep, 0.5 mM L-glutamine, and 5.8 μL/mL NaHCO₃. The cell suspension was pre-plated for 30 minutes at 37° C. to improve culture purity. The suspended cells were then filtered through a cell strainer with 100 μm pore size and centrifuged at 1000 rpm for 5 minutes. The pellet was resuspended in supplemented Neurobasal medium. The cells were plated at different densities depending on the experiment. After 24 hours, the medium was replaced with serum-free neuronal culture medium.

### CSPG Coating

Glass coverslips were coated in poly-D-lysine (10 μg/mL) in water and incubated for 2 hours at 37° C. PDL solution was removed, coverslips were washed 3× with sterile water, and were left to dry at RT. They were then coated in an CSPG (1 μg/mL) and laminin (10 μg/mL) solution in HBSS for 2 hours at 37° C. until the time of seeding.

### CSPG Spot

A CSPG spot assay was made. A solution of CSPG (10 μg/mL) and laminin (10 μg/mL) was made in HBSS. Two 2 μL drops of the CSPG/laminin solution was placed on pre-coated PDL coverslips and let air dry for 1 hour. Coverslips were washed and incubated in Neurobasal at 37° C. until the time of seeding. After one day of culture, cells were treated with either sliders or peptides. For slider treatment, all media was removed and 20 μL of PA material was added directly on top of cells, followed by addition of fresh media.

### Scratch Assay

DIV7 neurons grown on PDL coverslips were scratched with a P200 pipette tip in two separate areas, and the coverslip was washed with Neurobasal media. They were then treated with CSPG (3 μg/mL) with or without sliders and peptides in media. Images were acquired on an Essen Bioscience IncuCyte S3 every 6 hours for 72 hours.

### Immunocytochemistry

Neurons cultured on glass coverslips were fixed in 4% paraformaldehyde (PFA) for 15 minutes. Coverslips were then washed twice with 1× PBS and twice with PBST (0.2% Triton in 1× PBS), followed by a 1-hour incubation in blocking buffer (10% NHS, 0.2% Triton in 1× PBS). The coverslips were incubated in primary antibody solution prepared in blocking buffer overnight at 4° C. Primary antibodies used were β-III tubulin (1:1000), CS56 (1:250), PTPσ (1:150), synaptophysin (1:500), and PSD-95 (1:1000). The next day, they were washed three times with PBST for 15 minutes each, followed by a 2-hour incubation in secondary antibody solution prepared in blocking buffer. The coverslips were then washed three times in PBS and immediately mounted with Immuno-Mount on a glass slide. The samples were stored at 4° C. until imaging. Low magnification images and confocal images were acquired.

### Uptake Assay

Neuro-2a cells were seeded on PDL-coated glass-like polymer bottom cell culture plates. The media was switched to 1% FBS at DIV1, and at DIV2 the cells were treated in DMEM without FBS. Fluorescent sliders were prepared using 3.125 mol % fluorescently labeled peptide and E2 PA. For slider addition, all media was removed and 20 μL PA was added directly on top of the cells. Media was replaced and incubated for 72 hours. The cells were then stained with 10 μM CellTracker blue for 1 hour at 37° C., and the solution was replaced with fresh DMEM. Live cell imaging was conducted at 37° C. supplying 5% CO₂.

### Example 4

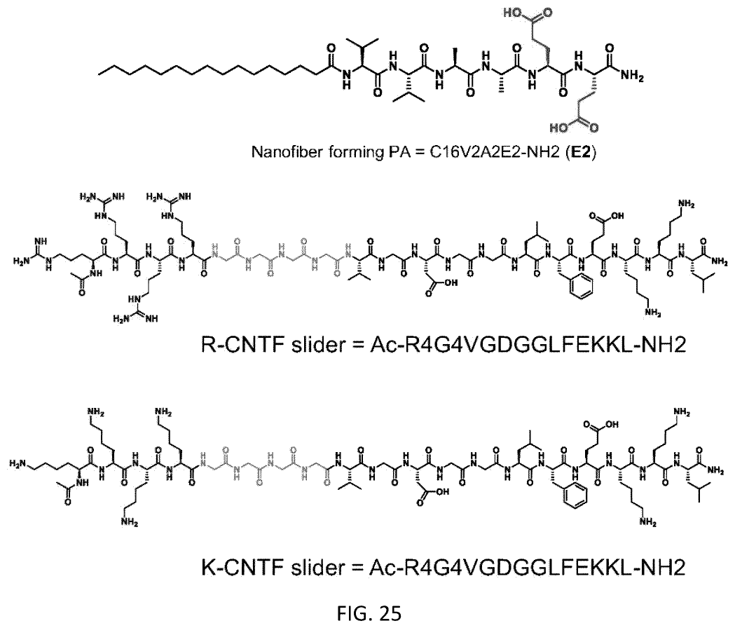

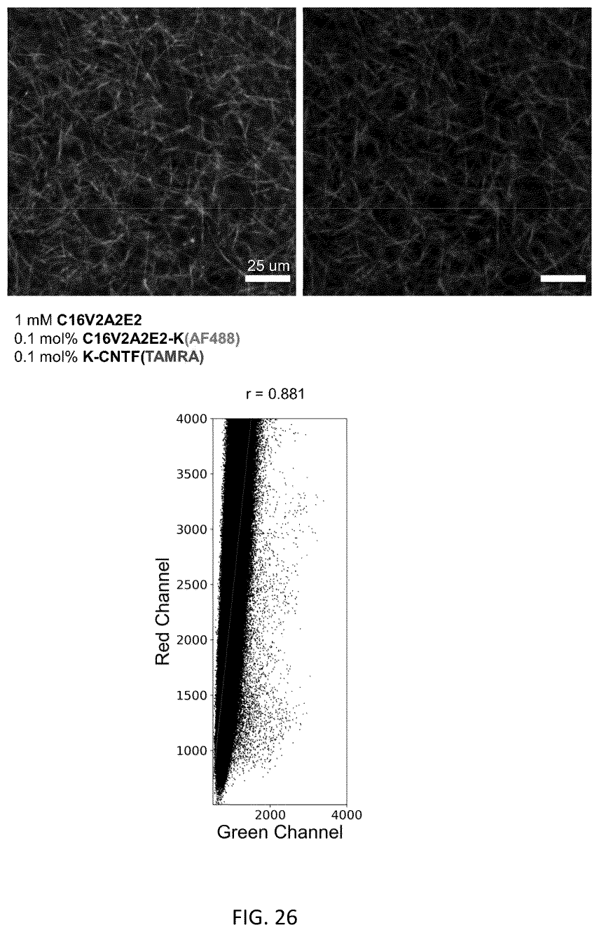

Slider systems containing a CNTF mimetic slider peptide were generated. Structures of representative CNTF slider systems are shown in FIG. 25. The CNTF mimetic sequence VGDGGLFEKKL (SEQ ID NO: 4) was used on a backbone PA C₁₆-V₂A₂E₂ (SEQ ID NO: 17). Two different sliders were generated as shown, referred to as R-CNTF and K-CNTF. Binding of the slider to the PA backbone was confirmed by NMR spectroscopy. Binding of the slider to the PA backbone was further verified by confocal fluorescence microscopy, as shown in FIG. 26, which shows nearly complete colocalization of the fluorescently labelled sliders and peptide amphiphile nanofibers.

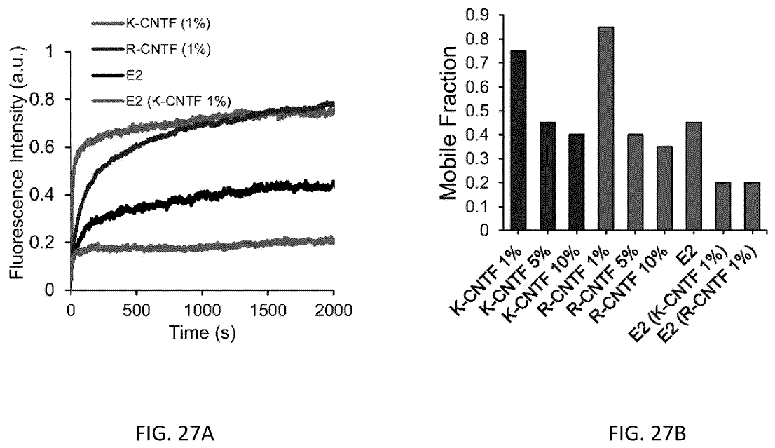

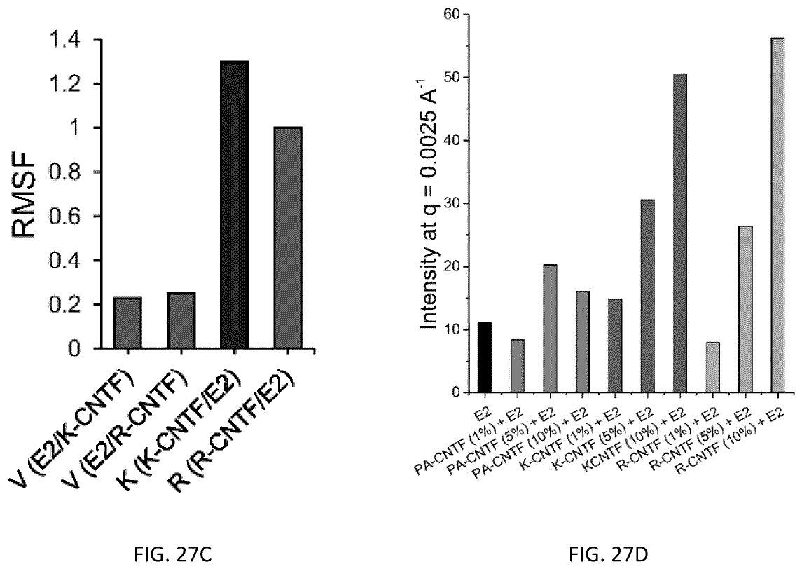

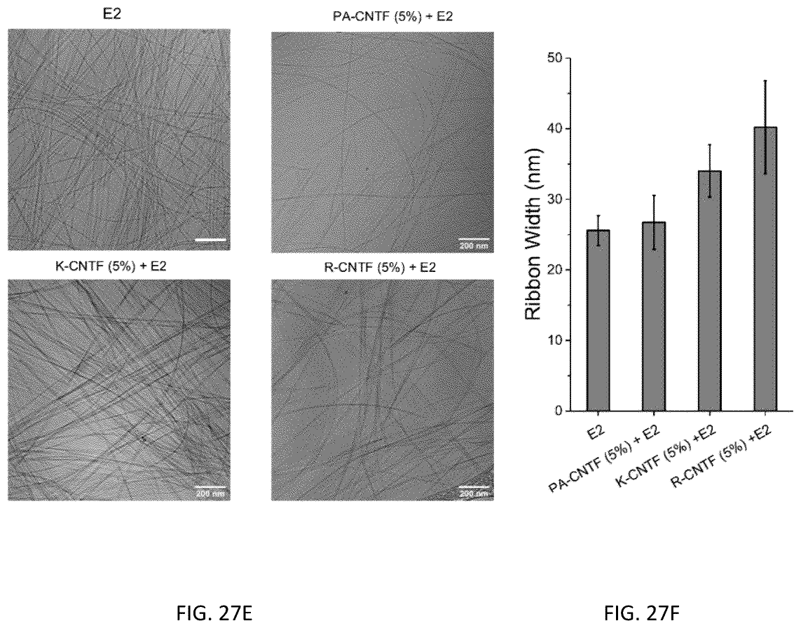

FRAP experiments were conducted to assess slider mobility. Results from FRAP are shown in FIG. 27. FIG. 27A shows fluorescence intensity values, demonstrating that sliders (K-CNTF, R-CNTF) were recovered faster than PA alone (E2 ), showing higher motility. PA in the presence of slider shows slower and lower recovery, supporting that while the slider is more motile, the PA nanofibers are stabilized by the slider and there are substantially less micelles. As per design, K-CNTF moves faster than R-CNTF. FIG. 27B shows the mobile fraction from FRAP experiments. Sliders have a larger mobile fraction. The mobile fraction of the slider decreases when their concentration increases, due to induction of bundling of PA nanofibers. The mobile fraction of E2 in the presence of the slider is substantially reduced. FIG. 27C shows RMSF data from atomistic molecular dynamic simulation. RMSF of valine residues of E2 is considerably lower than the RMSF of K or R residues in the slider, showing slider mobility in the simulation.

The stabilization of nanofibers by the slider was further observed by SAXS, as shown in FIG. 27D, which shows an increased in scattering indicating that sliders stabilize nanofibers, shifting the micelle/nanofiber equilibrium towards the nanofiber. FIG. 27E shows images demonstrating that sliders stabilize the nanofibers and induce transition to a ribbon-like morphology with increased width compared to E2 nanofibers alone. Results are quantified in FIG. 27F.

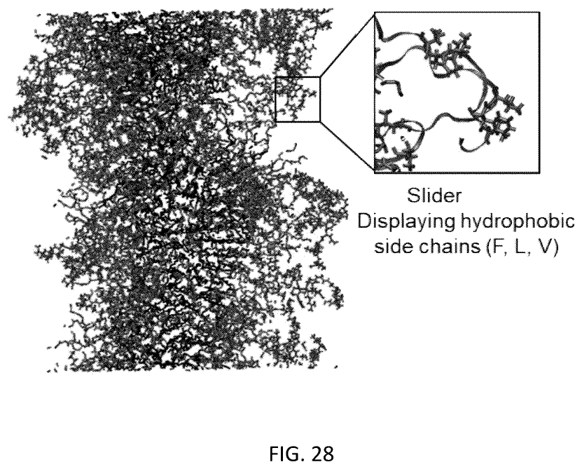

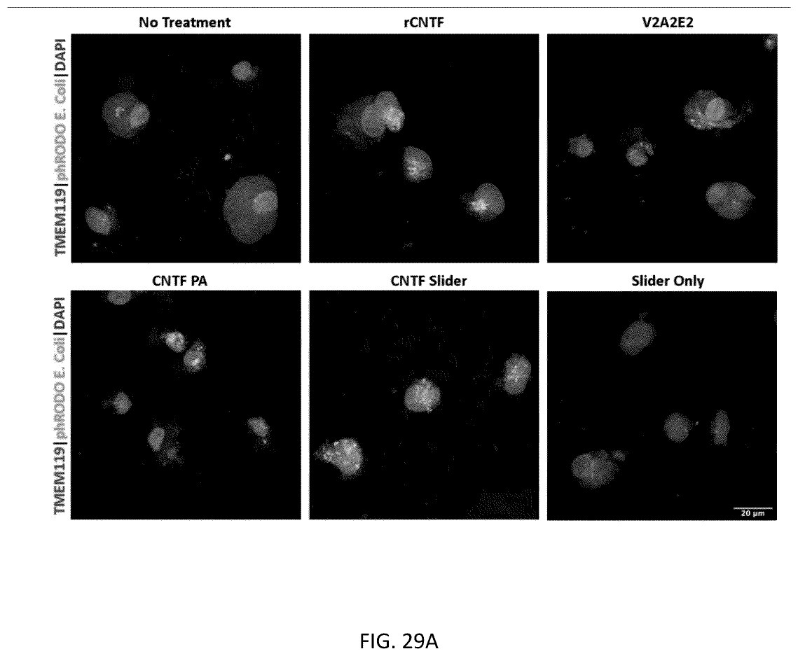

Results from experimental simulation and NMR spectroscopy demonstrate that the interaction of the slider with the PA backbone also allows the slider to enable a more bioactive conformation, displaying hydrophobic side chains towards the outside of the fiber (e.g. ready to interact with the receptor), as shown in FIG. 28.

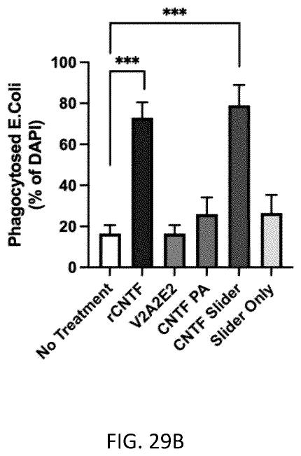

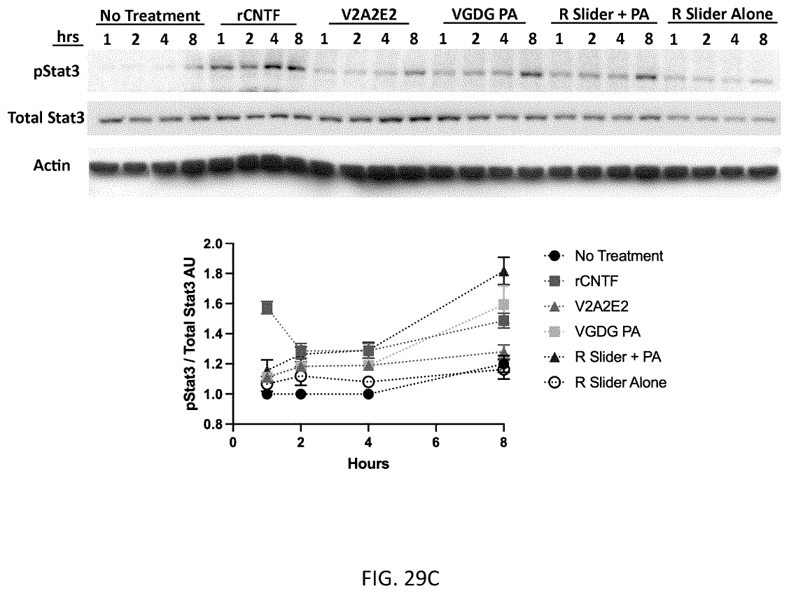

CNTF enhances microglial phagocytosis, engulfing and removing debris within the central nervous system. The R-CNTF slider was used in experiments to assess induction of microglial phagocytosis. Microglial cells were treated with no treatment, recombinant CNTF as a positive control, E2 alone, the R-CNTF slider, or the CNTF mimetic peptide alone (slider only). Results in FIG. 29 demonstrate that the R-CNTF slider enhanced phagocytosis and that levels were higher in the R-CNTF Slider compared to the CNTF mimetic peptide alone (slider only), demonstrating that interaction of the slider with the PA is significant for bioactivity and translation of signaling to a functional level (e.g. to induce phagocytosis).

CNTF activates the phosphorylation of STAT3, an essential transcription factor for neuronal survival, neuroprotection, and anti-inflammation. Activation with recombinant CNTF typically occurs within minutes to an hour, yet does not have long-lasting effects. Additional experiments were conducted to evaluate the CNTF slider system would prolong this activation. Microglial cells were grown to 70% confluency before being treated with materials for 1, 2, 4, and 8 hours. Lysates were collected in RIPA buffer with protease and phosphatase inhibitors, then quantified using a BCA assay. Proteins were separated by SDS-PAGE and transferred onto a PVDF membrane. The membrane was blocked with 5% BSA in TBST (TBS+0.1% Triton) for 30 minutes, followed by overnight incubation in primary antibodies diluted in blocking buffer. Membranes were then washed three times for 5 min each in blocking buffer, followed by incubation with secondary antibodies in blocking buffer for 45 min at room temperature. Membranes were washed three times, then protein signals were detected using Radiance Bioluminescent ECL substrate. Protein expression was quantified using ImageJ and normalized to Actin.

Signaling dynamics of CNTF-mimetic slider using Western Blot. Microglial cells were treated with either recombinant CNTF (rCNTF), C16V2A2E2 PA (V2A2E2), CNTF-mimetic PA, CNTF-mimetic slider+PA (R slider+PA), or CNTF-mimetic slider alone for 1, 2, 4, or 8 hours. Results are shown in FIG. 29C. Treatment with rCNTF peaked at 1 hr but decreased at later timepoints. Both the PA alone and slider alone showed little activation of Stat3. While the CNTF-mimetic slider with PA showed little activation at early timepoints, it had the strongest effect at 8 hours compared to all other groups. Additionally, there was a stronger effect of the slider with PA compared to the CNTF-mimetic PA. The dynamic nature of the R-slider system can improve upon the bioactivity of CNTF-mimetic sequences compared to covalent linking of the peptide sequence to a PA monomer.

### Example 5

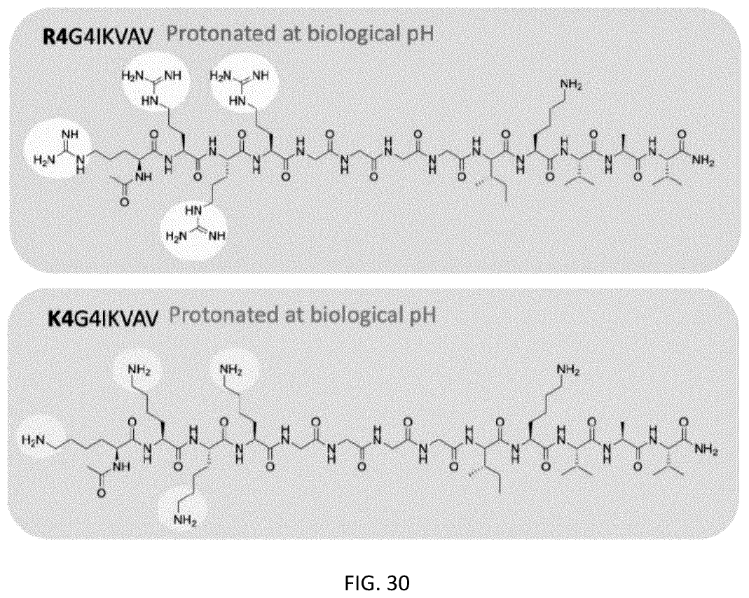

Slider systems containing the biomimetic sequence IKVAV were generated. The structure of exemplary sliders is shown in FIG. 30. The sliders contained the backbone PA C16-V₂A₂E₂ (SEQ ID NO: 17) and a slider peptide containing the biomimetic sequence IKVAV (SEQ ID NO: 7) and the charged sequence RRRR (SEQ ID NO: 20) or KKKK (SEQ ID NO: 21). The charged sequence and the biomimetic sequence were connected by a spacer containing 4 glycine residues (GGGG (SEQ ID NO: 18)).

Prior studies have demonstrated that IKVAV-functionalized peptide amphiphiles can enhance neural regeneration, particularly in spinal cord injury models. However, this study introduces a fundamentally different approach by using a dynamic slider system rather than covalent attachment of the bioactive sequence to the fiber backbone. This distinction allows for independent control over material stability and bioactive sequence mobility, which is not achievable with conventional peptide amphiphile systems. The ability to separate fiber movement from bioactive epitope movement provides a novel strategy for dynamically tuning biomaterial-cell interactions.

The primary objective of this research was to develop a dynamic self-assembling nanomaterial system utilizing the IKVAV signaling sequence to promote neuronal outgrowth and enhance integrin expression in hNPCs. The key findings demonstrate that the incorporation of both RIKVAV and KIKVAV sliders at specific concentrations (10% and 50%) on E2 fibers resulted in notable differences in material morphology and function. Bundling behavior was observed across all slider concentrations, with larger, more pronounced bundles forming at higher concentrations of both R and K sliders. However, biophysical characterization revealed greater dynamic mobility in the arginine-containing (R) system compared to the lysine-containing (K) system, which led to R being prioritized for biological experiments.

In biological assays, the RIKVAV slider system resulted in significant integrin upregulation, increased neuronal differentiation, and longer neurite outgrowth, particularly at the R50% concentration. This suggests that the positive charge of the RIKVAV sequence enhances the dynamic presentation of the bioactive epitope, which in turn contributes to its biological effects. While the E2 nanofiber scaffold itself exhibits dynamic movement, this alone would not account for the observed biological outcomes without the presence of a functional bioactive IKVAV epitope.

Biophysical characterization further supported the role of dynamic mobility in enhancing bioactivity. FRAP experiments demonstrated that slider movement was most pronounced at R50%, indicating greater molecular mobility of the bioactive sequence in this condition. Here, “optimal” refers to the highest observed rate of slider mobility, which is hypothesized to correlate with increased cellular responsiveness. FRET confirmed binding interactions between the sliders and the fibers, but it did not directly assess molecular mobility. Together, these results indicate that the ability of the slider to move along the fiber surface enhances bioactive sequence presentation and cell signaling.

The upregulation of integrin observed in immunostaining experiments provides additional evidence for bioactivity through β1-integrin signaling. The bright, punctate integrin staining observed in the E2+R50% treatment group suggests recruitment of integrin into lipid rafts, which are known to facilitate focal adhesion formation and neurite outgrowth. This confirms that the bioactivity of IKVAV (e.g. in promoting neuronal differentiation and survival through integrin activation) and is enhanced within the slider system.

These findings have significant implications for next-generation biomaterials for neural regeneration. By employing a dynamic system where the bioactive sequence is mobile, this study demonstrates that greater biological activity can be achieved through enhanced epitope presentation and mobility. The bundling behavior observed in E2+R50% samples mirrors hierarchical ECM-like properties, demonstrating that these materials could be used as scaffolds for neuronal growth or other regenerative applications. Furthermore, the increased integrin expression observed in hNPCs treated with these materials suggests potential applications for nerve repair and implantable scaffolds.

### METHODS

Materials used were PBS buffer, C16-V2A2E2 (E2 ), C16-V2A2E2-K (TAMRA) (E2-TAMRA), C16-V2A2E2-AlexaFluor488, Ac-R4G4IKVAV (referred to as “RIKVAV”), Ac-K4G4IKVAV (referred to as “KIKVAV”), Ac-R4G4IKVAV-K (TAMRA), and Ac-K4G4IKVAV-K (TAMRA).

The peptides and PAs were synthesized using standard Fmoc solid-phase peptide synthesis. The molecular peptide sliders were purified by reverse-phase high performance liquid chromatography (HPLC) under acidic conditions (0.1% TFA in water/acetonitrile) with a gradient ramp from 2% to 50%. C16-V2A2E2 was purified under basic conditions (0.1% ammonium hydroxide in water/acetonitrile) using the same gradient. Purity and molecular weight were confirmed by liquid chromatography-mass spectrometry (LC-MS), and all purified peptides were lyophilized and stored as dry powders until use.

### Slider Preparation

Lyophilized PA powder was dissolved in PBS at either 2 mM or 10 mM and pH adjusted to ˜7.4 by adding 1 M NaOH. Samples were bath sonicated for 30 minutes, then annealed in an 80° C. water bath for 1 hr, and slowly cooled overnight to room temperature (RT). Annealing at 80° C. for 1 hr was performed to promote self-assembly of PAs into nanofibers. For fluorescently labeled PA solutions, E2 dissolved in PBS was mixed with 0.1 mol % E2-TAMRA in PBS, pH adjusted to ˜7.4 with 1M NaOH, sonicated, and annealed. PA solutions were further diluted in PBS and vortex mixed for ˜20 s. Lyophilized peptides were dissolved in water at 1 mM. Slider solutions were prepared by adding peptides to the E2 solution, 5 μL at a time, to the wall of the Eppendorf tube. Solutions were immediately vortex mixed ˜20 s after each addition.

### qPCR/Melting Temperature

Fluorescence measurements were conducted using a CFX96 Touch Real-Time PCR Detection System by Bio-Rad. The ROX channel was employed to measure fluorescence, with ROX serving as a reference dye for fluorescence detection. Nile red, having similar excitation and emission wavelengths to ROX, was used as the fluorophore for this experiment. Fluorescence readings were recorded at one-minute intervals across the entire temperature range of 25° C. to 99° C., with a heating rate of 1° C. per minute.

To the experimental samples, 2 μL of 100 μM nile red in ethanol was added, yielding a final volume of 70 μL. The fluorescence measurements were taken as the temperature increased. As the fibers within the samples melted, nile red was released into the solution, resulting in a decrease in fluorescence. In order to visualize the melting points within the data, the first derivative of the fluorescence signal with respect to temperature (−d(RFU)/dT) was calculated, and the negative sign was applied to ensure that the peak representing the melting transition appeared as a positive value. This derivative analysis aided in identifying the critical points within the melting curve data.

### Fluorescence Recovery after Photobleaching (FRAP)

A 2 mM stock solution of E2 was prepared. Samples were diluted with PBS to achieve a final concentration of 500 μM E2. Different molar percentages of IKVAV peptides were added. When labeled peptides were used, 1 mol % of the fluorescent peptide was added to the sample, with the remaining molar percent peptide added being unlabeled. On a glass slide, a silicon spacer with four wells was applied. In each well, approximately 10 μL of the sample was pipetted and a glass coverslip was used to seal the wells. The sample was allowed to equilibrate for 15 minutes and then examined on a Nikon confocal laser scanning microscope approximately 100 μm from the surface of the coverslip with laser power at 0.1. Then, three 15 μm ROIs were selected in relatively homogenous sites and a FRAP experiment was run with 15 s for initial acquisition, 1 s for bleaching, and 1 hr for post-bleaching, with acquisition every second. The file was then bleach-corrected in ImageJ and the recovery over time of the three ROIs were averaged and plotted. Averaging the recovery of multiple ROIs minimizes the impact of local heterogeneities and provides a representative measure of diffusion dynamics across the sample.

### Circular Dichroism (CD) Spectroscopy

CD spectra were acquired in PBS at a concentration of 75 μM for the PA. The sliders were used at 10 mol %. The slider concentration of 10 mol % was chosen based on previous studies indicating optimal secondary structure formation at this ratio. CD spectra were recorded on a JASCO model J-850 spectropolarimeter using a quartz cell. Continuous scanning mode was used with a scanning speed of 100 nm per minute with the sensitivity set to standard mode. High Tension (HT) voltage was recorded for each sample to ensure that the measurement was not saturated. An accumulation of three measurements was used and a buffer sample was background-subtracted to obtain final spectra.

### Small/Wide Angle X-Ray Scattering (SAXS/WAXS)

SAXS and WAXS experiments were performed at beamline 5-ID-D of the DuPontNorthwestern-Dow Collaborative Access Team (DND-CAT) Synchrotron Research Center at the Advanced Photon Source, Argonne National Laboratory. A sample solution (100-150 μL, [PA]=0.5 w/v % (˜5 mM)) prepared in PBS buffer was loaded and irradiated with X-rays for an exposure time of 0.5, 2, 3, or 10 seconds. Data was collected with an X-ray energy at 17 keV (λ=0.83 Å) with a triple-area detector system. The scattering intensity was recorded in the interval 0.002390 &1t; q &1t; 4.4578 Å −1. The wave vector q is defined as (4π/λ) sin (θ/2), where θ is the scattering angle. The acquired 2D scattering data were then reduced to 1D intensity vs. wave vector plots via azimuthal integration around the beam center in GSAS-II software. Background scattering patterns were obtained from samples containing PBS. This background data was then subtracted from experimental data. SAXS was used to characterize the mesoscale structure and fiber bundling, while WAXS provided insights into the molecular arrangement and β-sheet content within the E2 nanofibers. All data was analyzed using the OriginLab software package.

### Förster Resonance Energy Transfer (FRET)

FRET spectroscopy and microscopy were performed. This technique relies on using two fluorescent labels with overlapping emission (donor) and absorbance (acceptor). The self-assembling PA nanofibers labeled with AlexaFluor488 emit in the green and act as donors (520 nm), and the peptide signaling sequence labeled with TAMRA emits in the orange red and will act as the acceptor (560 nm). AlexaFluor488 and TAMRA have been chosen for their photophysical properties. Based on how close the two fluorescent probes are, the acceptor will quench the emission of the donor and the energy transfer will cause emission from the acceptor by exciting the donor. If the PA and peptide slider are within about 10 nm of each other, they will affect the fluorescent read out of the PA, and this exact difference can be used to calculate their distance from each other. To collect data, the fluorescence emission of each fluorophore was measured using a plate reader across a range of excitation wavelengths. The efficiency of the energy transfer is proportional to the Förster radius (the distance at which efficiency is 50% according to equation: R₀=[2.8×10¹⁷×κ²×Q_{D}×E_{A}×J(λ)]^{1/6} nanometers), where R₀ is the distance between the probes, κ² is the orientation factor between the fluorescent dipoles, Q_{D} is the donor quantum field, E_{A} is the maximal acceptor extinction coefficient in reciprocal moles per centimeter, and J(λ) is the spectral overlap integral between the normalized donor fluorescence. Using this equation, the distance between the two probes can be determined down to the nanometer by measuring energy transfer efficiency.

### Cryo-Transmission Electron Microscopy (Cryo-TEM)

Copper grids (300-mesh) with a lacey carbon film (Electron Microscopy Sciences) were glow-discharged for 30 s using a PELCO easiGlow system (Ted Pella, Inc.). PA solutions were prepared by diluting to 1 mM in water immediately prior to use. A 7 μL aliquot of the PA solution was applied to the grids, blotted, and plunge-frozen in liquid ethane using an FEI Vitrobot Mark IV (FEI) under 95-100% humidity. Vitrified samples were then transferred, under liquid nitrogen, to a Gatan 626 cryo-holder (Gatan) and imaged using a JEOL 1230 TEM at an accelerating voltage of 100 kV. During imaging, liquid nitrogen temperatures were maintained, and micrographs were recorded with a Gatan 832 CCD camera. Bundle morphology was qualitatively observed across images of each sample.

### E16 Primary Mouse Cortical Neuron Dissection and Culture

Primary cortical neurons were isolated from embryonic mouse brains. Briefly, a time-pregnant CD1 mouse was euthanized by cervical dislocation, and embryos were collected on embryonic day 16 (E16). Cerebral cortices were carefully dissected, and meninges were removed. The cortices were transferred into Hank's Balanced Salt Solution (HBSS) containing 1% penicillin-streptomycin (pen-strep) and then digested in 0.25% Trypsin/EDTA with DNAse for 10 minutes at 37° C. After enzymatic digestion, the tissue was mechanically dissociated and allowed to settle. The supernatant was collected, centrifuged at 1000 rpm for 5 minutes, and the resulting pellet was resuspended in CO2-equilibrated Neurobasal medium supplemented with 10% normal horse serum (NHS), 1% pen-strep, 0.5 mM L-glutamine, and 5.8 μL/mL NaHCO₃. To enhance culture purity, the cell suspension was pre-plated for 30 minutes at 37° C. The suspension was then filtered through a 100 μm pore cell strainer and centrifuged again at 1000 rpm for 5 minutes. The pellet was resuspended in supplemented Neurobasal medium, and cells were plated at experiment-specific densities. After 24 hrs, the medium was replaced with a serum-free neuronal culture medium.

### Primary Cortical Neuron In Vitro Assays

Neurons were seeded onto poly-D-lysine (PDL) coated surfaces and treated with PA in media for all in vitro assays. Glass coverslips were coated with a solution of 0.01 mg/mL PDL in sterile Milli-Q water for at least 2 h at 37° C., then washed twice with sterile Milli-Q water and left to dry overnight. For PA treatment in media, PA solutions were diluted to the desired concentration in cell media immediately prior to treatment. The concentration of the treatment was calculated based on the molarity of the fiber. The concentration of the slider is always in relation to mol % concentration of fiber.

### Differentiation of iPSCs into hNPCs

Induced pluripotent stem cells (iPSCs) were differentiated into human neural progenitor cells (hNPCs) through a three-phase protocol: neuralization, differentiation, and maturation. On Day 0, cells were cultured in a neural induction medium comprising DMEM/F12 (25 mL), Neurobasal (25 mL), B27 supplement (500 μL), N2 supplement (250 μL), GluMAX (500 μL), and non-essential amino acids (NEAA: 500 μL). Retinoic acid (1 μM), SAG (1 μM), SB431542 (10 μM), and LDN193189 (100 nM) were added to promote neuralization. Cells were passaged using Accutase as needed, with daily media changes. On Day 6, cells were transitioned to a differentiation medium consisting of DMEM/F12 (25 mL), Neurobasal (25 mL), B27 supplement (500 μL), N2 supplement (250 μL), GluMAX (500 μL), and NEAA (500 μL), supplemented with retinoic acid (1 μM), SAG (1 μM), DAPT (5 μM), and SU5402 (4 μM). Cells were passaged using TrypLE Express with DNase I (1:10) and maintained with daily medium changes. On Day 14, cells were matured in a medium containing Neurobasal (50 mL), B27 supplement (500 μL), N2 supplement (250 μL), GluMAX (500 μL), NEAA (500 μL), penicillin-streptomycin (500 μL), and fetal bovine serum (500 μL). Ascorbic acid (0.2 μg/mL), brain-derived neurotrophic factor (10 ng/ml), ciliary neurotrophic factor (10 ng/ml), and glial cell line-derived neurotrophic factor (10 ng/mL) were added to support neuronal maturation. Media changes were performed daily.

### Human Neural Progenitor Cell (hNPCs) Culture

NPCs were derived from iPSCs following the protocol above. Cells were cultured on Matrigel-coated 6-well plates prepared by thawing 50 μL of Matrigel on ice and diluting it in 1 mL cold DMEM/F-12 with 15 mM HEPES. Plates were coated with 1 mL of this solution per well and incubated at RT for 1 hr before use. NPCs were passaged using Accutase; cells were detached, collected in DMEM/F-12, centrifuged at 300×g for 5 minutes, and resuspended in complete STEMdiff NPC medium. Cells were plated at a density of 1.25×10⁵ cells/cm² with Rock inhibitor (Y-27632) and maintained with daily medium changes using prewarmed STEMdiff NPC medium without Y-27632. Cells were passaged as needed or after reaching confluency.

### hNPC In Vitro Assays

NPCs were seeded onto poly-L-ornithine (PLO) and laminin-coated surfaces for all in vitro assays. Plastic tissue culture-treated 24-well plates were first coated with PLO at a concentration of 15 μg/mL, prepared in PBS. Each well received 500 μL of this solution, and the plates were incubated at RT for 2 hr or wrapped in parafilm and incubated overnight at 4° C. Wells were then washed twice with PBS. For laminin coating, a solution was prepared at 10 μg/mL in PBS, and 500 μL was added to each well. After a 2 hr incubation at RT or overnight at 4° C., wells were washed twice with PBS and left filled with PBS. hNPCs were seeded at a density of 40,000 cells per well in 500 μL of NPC differentiation media. The following day, 500 μL of BrainPhys kit media supplemented with 20 μM PA was added to each well. A ½ media change was performed every 2-3 days, with fresh BrainPhys media containing 10 μM PA added to maintain treatment. hNPC differentiation was carried out using the BrainPhys hPSC Neuron kit (STEMCELL Technologies). Differentiation medium was prepared by combining 200 μL NeuroCult SMI supplement, 100 μL N2 Supplement-A, 2 μL of 100 μg/mL BDNF, 2 μL of 100 μg/mL GDNF, 50 μL of 100 mg/mL Dibutyryl-cAMP, and 4 μL of 0.5 mM ascorbic acid in 10 mL of BrainPhys medium. PA solutions were vortexed briefly before being added to the media to ensure uniformity. Cells were either imaged or fixed based on the experiment timeline. For western blot, cells were seeded at 400,000 cells per well, and associated treatment and coating volumes were scaled for a six-well plate.

### Immunofluorescence Staining

All steps were carried out at RT unless otherwise specified, with all washes conducted for 15 minutes with gentle shaking. Day 1: Cells were first rinsed briefly in 37° C. PBS after removing the culture media, followed by fixation with freshly prepared 4% paraformaldehyde (PFA) in 1× PBS at 37° C. for 15 minutes. Fixed cells were washed twice with PBS, then permeabilized in PBST (PBS+0.2% Triton X-100) for 15 minutes and blocked for 1 hr in 10% serum in PBST. Primary antibodies, diluted in 10% serum in PBST, were applied, and samples were incubated overnight at 4° C. with shaking. Day 2: After removing the primary antibodies, samples were washed three times in PBST. Secondary antibodies, DAPI (1:500), and Phalloidin (1:400) were diluted in 10% serum in PBST and incubated for 2 hrs at RT. protected from light. After three additional washes in PBS, coverslips were mounted onto glass slides with Immuno-Mount. Slides were allowed to dry overnight at RT in darkness, and further stored at 4° C. until imaging. Primary antibodies used were: 1:1000 βTubb3 gp, 1:250 aITGB1 ms, 1:800 Nestin rb, and 1:100 Pax6 ms. Secondary antibodies used were: 1:800 AlexaFluor 488 Ms, 1:800 AlexaFluor 647 Gp, and 1:800 Cy3 Rb. Any quantification of immunostaining images averaged values across a minimum of four images each from two slides that received identical treatment (eight total). Standard error was chosen to provide an estimate of the variability in the sample mean across independent images and replicates, reflecting precision. Fluorescence imaging was performed using a confocal microscope to visualize stained cellular structures with high resolution. Z-stack imaging was applied where necessary to capture cellular morphology in three dimensions, and maximum intensity projections were generated for analysis. Image quantification was conducted using ImageJ, with brightness and contrast uniformly adjusted across all samples. Any statistical analyses of immunofluorescence intensity, colocalization, or morphology were performed using standardized protocols to ensure consistency across experimental conditions.

### Live/Dead Viability Assay

A Live/Dead viability assay was conducted using the Invitrogen Live/Dead Kit (L3224) to assess cell viability. The positive control was treated with 70% ethanol for 5-10 minutes to induce cell death. A working solution containing 4 μM EthD-1 and 2 μM Calcein AM in DPBS was prepared. After removing the media, cells were washed with DPBS and incubated in 500 μL of the live/dead working solution at 37° C. for 20 minutes. The working solution was removed and fresh DPBS was added. Fluorescence was imaged using an Evos M5000 microscope, with green fluorescence indicating live cells and red fluorescence indicating dead cells. Data analysis was performed using ImageJ to determine viability, calculated as the ratio of live cells to total cells (live+dead). This metric was chosen because it provides a quantitative assessment of cell survival across treatment conditions, enabling comparisons of viability among groups. At least eight images, four from two separate wells were analyzed and averaged for each treatment condition. Statistical analysis for Live/Dead Viability data was performed using one-way ANOVA to compare cell viability among treatment groups.

### Scanning Electron Microscopy (SEM)

Samples were fixed with 2% PFA+2.5% Glyceraldehyde for 15 minutes, then washed twice with PBS. Then, they were dehydrated through a graded ethanol series, increasing through 30, 40, 50, 60, 70, 80, 90, 95, and 100% after 15 minutes in each, followed by critical point drying to prevent surface tension artifacts and remove ethanol. Once dried, the samples were mounted on stubs using carbon tape and coated with an 16 nm layer of osmium using an osmium coater (Filgen, OPC-60A) to ensure conductivity. Imaging was performed using a Hitachi SU8030 Scanning Electron Microscope (EPIC SEM) at an accelerating voltage of 2.5 kV. Both secondary electron (SE) and backscattered electron (BSE) modes were used to capture high-resolution images of surface morphology.

### Statistical Analysis

Data analysis was performed using GraphPad Prism (version 9.5.1), unless otherwise specified. A one-way ANOVA was chosen for comparisons among multiple groups based on the assumption of normal distribution and homogeneity of variance. For neuron length analysis, a Kruskal-Wallis test was used to account for the non-normal distribution of the data. The statistical tests and parameters applied for each experiment are detailed in the corresponding figure legends. A P-value of 0.05 or less (P≤0.05) was considered statistically significant. Significance levels are indicated in figures using asterisks: P≤0.05 (*), P≤0.01 (**), and P≤0.001 (***). For confocal and EVOS immunostaining data, error bars represent the standard error of the mean from at least 8 images obtained from two separate coverslips. SEM was used to reflect the variability of the mean across replicates and highlight precision. Unless otherwise stated, all error bars in the graphs indicate standard error of the mean.

### Results

FRET to Assess Binding. The material design relies on the assumption that the slider binds the surface of the fiber. To confirm this, FRET was performed. The slider was labeled with TAMRA, and the fiber was labeled with AlexaFluor488. The fiber serves as the donor (AlexaFluor488, excitation: 488 nm, emission: 520 nm), and the slider serves as the acceptor

(TAMRA, excitation: 560 nm, emission: ˜580 nm). When the donor molecule is excited, it can transfer energy to the acceptor molecule, quenching its own emission and exciting the acceptor instead. This is a distance-dependent phenomenon that occurs only when the two probes are less than 10 nm apart, confirming binding.

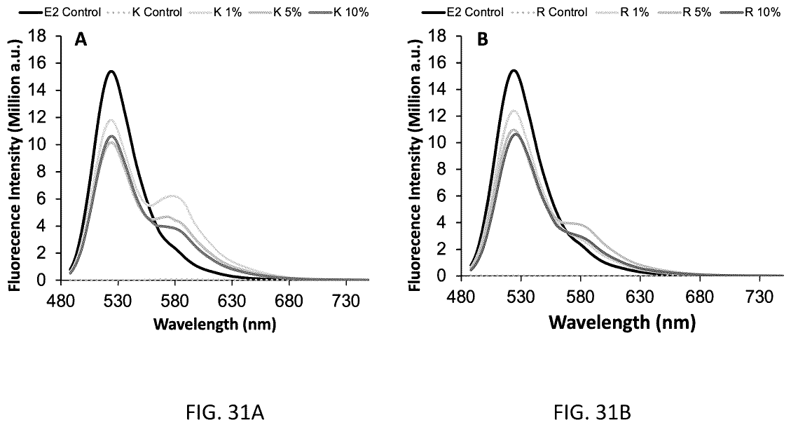

Results shown in FIG. 31 confirm binding between the slider and fiber for both the lysine and the arginine IKVAV slider variants, as shown by the characteristic quenching of donor fluorescence at 520 nm and the corresponding increase in acceptor emission at ˜580 nm. Notably, this peak at ˜580 nm is absent in the control sample containing only the donor-labeled E2 molecules, confirming that the energy transfer—and thus binding—requires the presence of the acceptor-labeled slider.

Confocal Imaging of Material and Bundles. The morphology of the slider system functionalized with the IKVAV slider sequence was visualized under a confocal microscope (FIG. 32) Typically, slider materials have a threshold concentration of bundling depending on the bioactive sequence used. In this context, bundling refers to the lateral aggregation of individual nanofibers into thicker, higher-order structures, often visible as bright, rope-like features under microscopy. This phenomenon is influenced by molecular interactions and epitope presentation, and is significant because it can impact the mechanical properties, cellular interactions, and diffusivity within the material. In slider systems containing different bioactive epitopes, this threshold concentration ranged from 5-10 mol % slider, with materials below this level appearing more homogeneous and unbundled. However, for sliders with the IKVAV sequence, bundling is exhibited at concentrations as low as 1 mol % sliders. To elucidate this bundling behavior, various slider concentrations were imaged under confocal, which indicated that bundles were a consistent characteristic of this material, and the degree of bundling is dependent on IKVAV slider concentration. A similar trend was seen with both RIKVAV and KIKVAV sliders, whereas E2 PAs without slider show no bundling morphology.

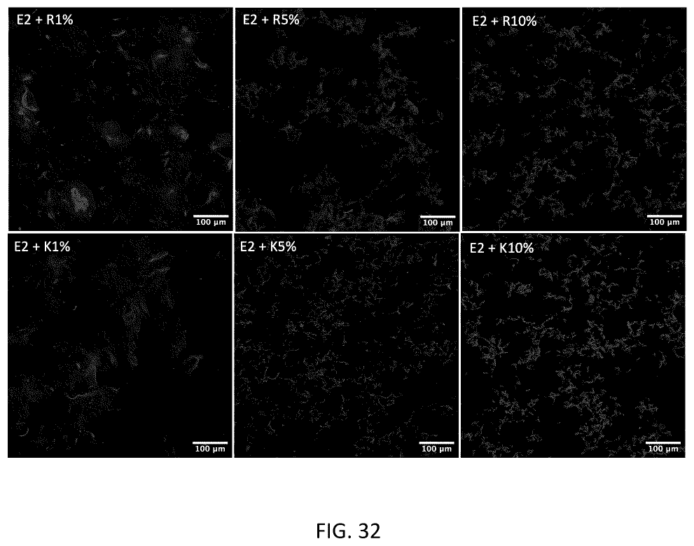

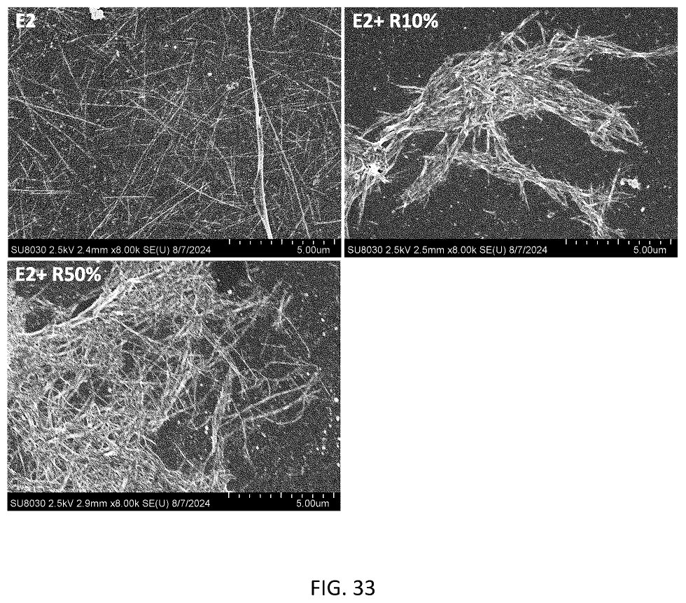

SEM Bundle Morphology. SEM was used for higher-resolution visualization of bundling (FIG. 33). This method allows individual fibers and their organization into bundles to be observed more clearly, and in cell-containing samples, it can reveal interactions between cells and the material surface. In the material samples, E2 fibers alone formed a relatively uniform, unbundled network across the coverslip. In contrast, the addition of IKVAV sliders resulted in the formation of dense, woven bundles. Notably, the E2+R50% sample exhibited larger and more pronounced bundles than the E2+R10% sample, suggesting a concentration-dependent effect on bundling. This difference in fiber organization may significantly influence how the material functions as a scaffold for neuronal growth and cell-material interactions.

CryoTEM of Fibers. To visualize the bundling on a smaller scale, CryoTEM was performed. This technique allowed the material to be observed on the scale of individual fibers with higher resolution (FIG. 34). Comparing just the fiber alone to the fiber with bound sliders, there are notable differences in fiber morphology. E2 fibers have characteristically long, uniform fibers with a slight ribbon twisting. This twisting can be seen in the repeating, spindle-shaped look of the fiber where the ribbon shape is twisted, displaying portions with various diameters. This twisting morphology is present when the slider is bound to the fiber, but as the concentration of the slider increases, the fiber length appears shorter. To further investigate, the dynamics of the nanofiber were assessed by FRAP.

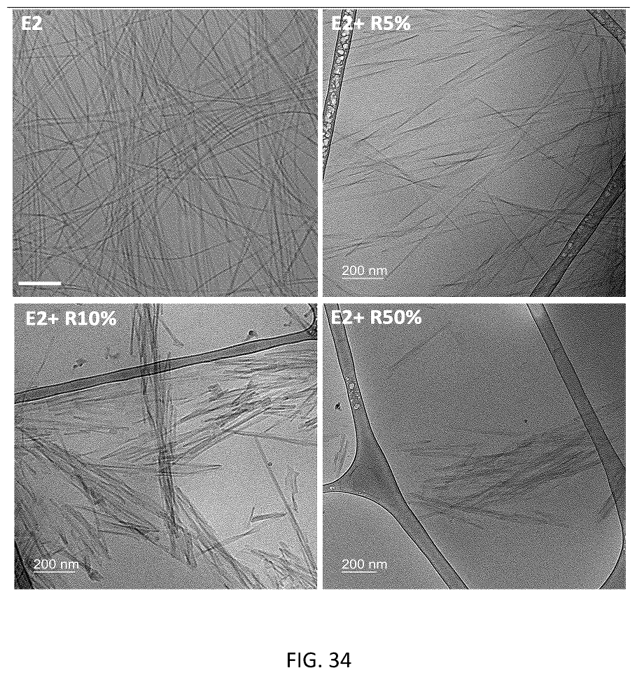

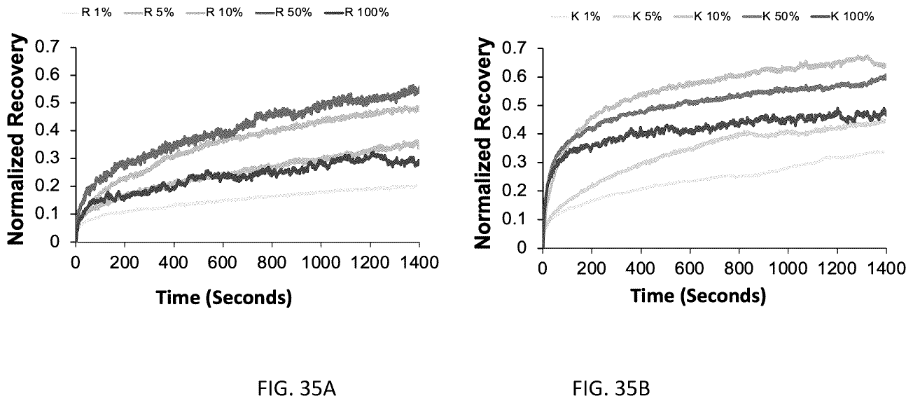

FRAP of Sliders. The dynamics of the slider on the nanofiber across various concentrations were assessed using FRAP experiments. The slider was fluorescently labeled, three regions were bleached, and their recovery was tracked over an hour. Recovery of fluorescence in the bleached regions indicates that unbleached slider molecules are moving into the bleached area, reflecting their mobility along the fiber surface. These values were then averaged and plotted across the various concentrations of arginine and lysine variants of the material. The slider motility was investigated first to identify the most dynamic concentration for later biology experiments (FIG. 35).

Despite the bundling of the material, there appears to be dynamic movement of the sliders. The total recovery observed for the arginine and lysine sliders was consistent with what was seen with other non-bundled slider materials. Additionally, each material has an apparent “optimal” concentration where it displayed the greatest recovery, and therefore the highest mobility. For the arginine material, this concentration is 50 mol % slider to fiber, and for the lysine material, it is 10 mol % slider to fiber. Greater dynamics are correlated with greater bioactivity in signaling materials, as such these are the proposed optimal concentrations.

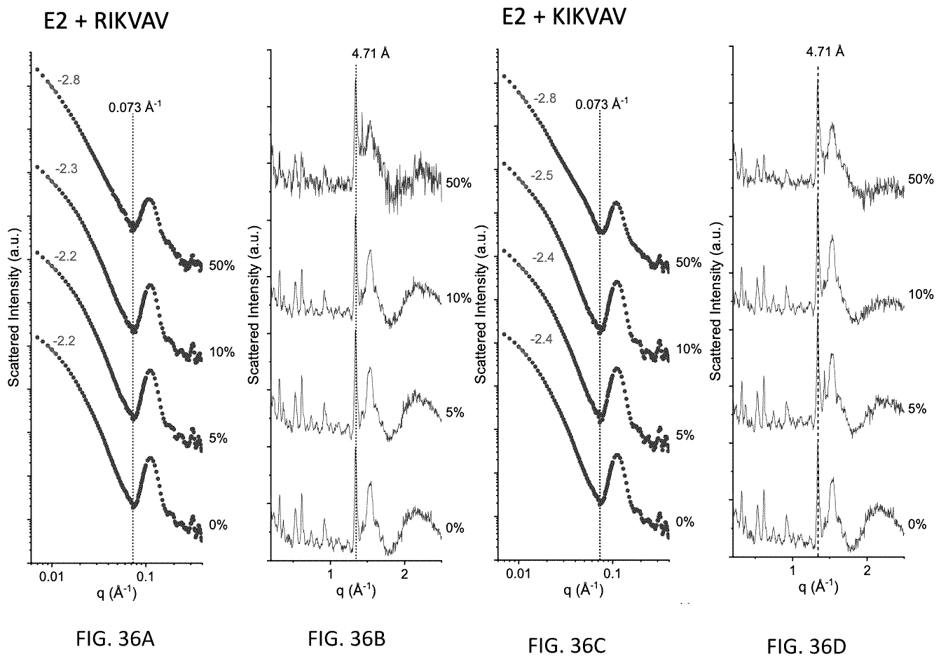

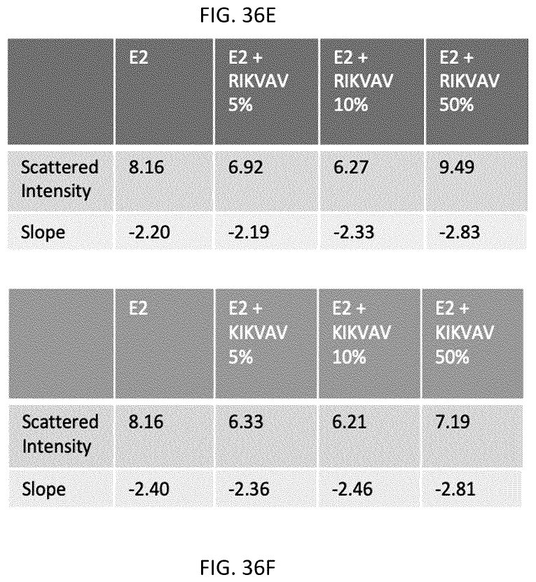

SAXS and WAXS. To establish differences in nanofiber morphology when IKVAV sliders are bound to PA nanostructures, SAXS and WAXS were performed on varying slider concentrations. The SAXS data in FIG. 36A and FIG. 36C reveal key differences in the structural organization of E2 fibers functionalized with RIKVAV and KIKVAV sliders. At low q values, the slope of the SAXS profiles indicates changes in the width and aggregation of the fibers. For RIKVAV-sliders (FIG. 36A), the slope increases from −2.2 at 0% sliders to −2.8 at 50% sliders, suggesting a transition to wider and possibly aggregated nanofibers with increasing slider concentration. Similarly, KIKVAV sliders (FIG. 36C) show a slope increase from −2.4 to −2.8, although the change is less pronounced, suggesting differences in the bundling mechanism or interactions of lysine-based sliders compared to arginine-based ones. The negative form factor peak near q=0.073 A⁻¹ in all SAXS profiles suggests the thickness of the nanofibers remains stable upon addition of sliders. This feature becomes sharper with increasing slider concentration, indicating a more regular structure as the slider concentration increases.

The WAXS data in FIG. 36B and FIG. 36D provide insight into the molecular arrangement within the fibers. The sharp peak at q=4.71 A⁻¹ corresponds to β-sheet packing, a hallmark of E2 fibers. In both RIKVAV and KIKVAV systems, the intensity of this peak increases with slider concentration, reflecting enhanced β-sheet content and molecular ordering. However, the β-sheet signal in the RIKVAV system is more pronounced at 50% slider concentration compared to KIKVAV, suggesting that arginine-based sliders enhance β-sheet stability and ordering more effectively. These results confirm that fiber integrity is maintained in the presence of sliders, as β-sheet domains remain intact, maintaining nanofiber stability.

Together, these results highlight the role of slider concentration and sequence in modulating the structural and molecular properties of E2 fibers. The arginine-based RIKVAV sliders promote wider, more aggregated nanofibers and higher β-sheet content, whereas the lysine-based KIKVAV sliders result in less pronounced structural changes, likely due to differences in electrostatic or steric interactions.

Melting Temperature to Assess Stability. To see how the formation of bundles and the introduction of sliders change the material stability, the melting temperature of the fiber was assessed. This was done by intercollating the fiber monomers with nile red, then heating the samples in a qPRC machine and measuring the fluorescence. When the fiber melts, the nile red is released and adheres to the side of the qPCR tube and is no longer measured in the fluorescence intensity, indicating the melting point.

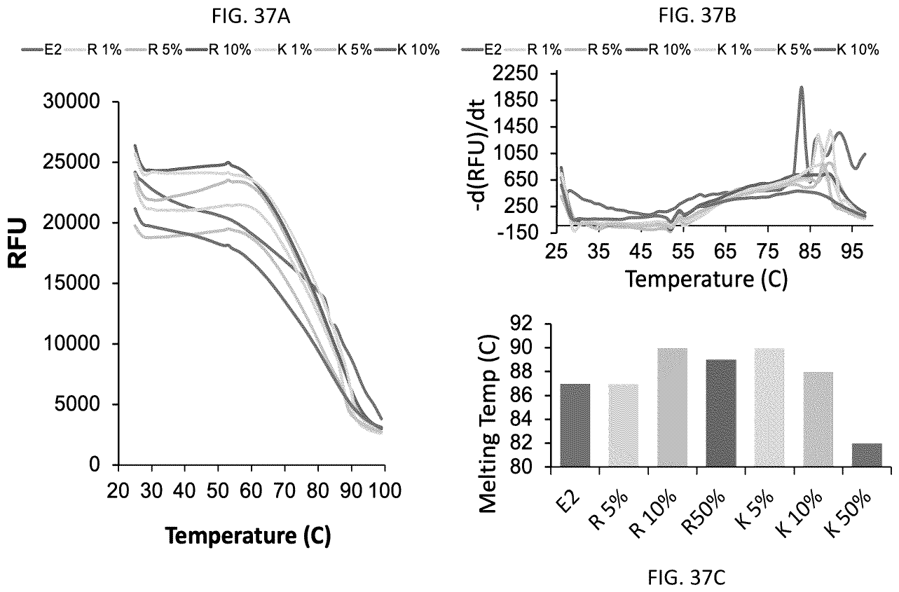

Across the tested conditions, E2 alone exhibited a melting point around 87° C. The introduction of R or K slider variants did not result in significant shifts in melting temperature no condition deviated by more than a few degrees (FIG. 37). A slight downward trend was observed in the lysine slider samples as slider concentration increased, but the change was modest. These results suggest that, within the tested concentration range, neither arginine nor lysine sliders meaningfully disrupt the thermal stability of the core nanostructure. Instead, the effects of the sliders appear to be more pronounced in supramolecular behavior—such as bundling or mobility—rather than in destabilizing the β-sheet structure that governs fiber assembly.

### Biological Studies

Determining the Model System. Based on preliminary material characterization, the arginine (R) slider variants demonstrated stronger binding affinity to the fiber and more consistent dynamic behavior compared to lysine (K) sliders. Given their superior performance in structural and FRET assays, subsequent biological experiments were conducted exclusively with R sliders to maximize the likelihood of observing functional cellular responses.

Experiments were performed using mouse primary cortical neurons and 2D dissociated organoid cultures to evaluate cellular responses to the material. To better mimic human biological systems and assess long-term effects compared to mouse cortical neuron cultures, the biological activity of the material was further investigated in human neural progenitor cells (hNPCs), a human stem cell line derived from induced pluripotent stem cells (iPSCs). This allowed for a translational approach to studying the effects of the IKVAV slider system on neural differentiation and integrin signaling.

### Study of IKVAV Sliders on Primary Mouse Cortical Neurons

Morphological Evaluation on Primary Mouse Cortical Neurons. To assess the material's ability to signal neurons and serve as a scaffold, primary mouse cortical neurons were treated with either coated coverslips or topically applied material. Coating the coverslip allows the slider material to act as an anchored base for neuron attachment and extension, whereas topical treatment exposes neurons to the sliders in solution while controlling for culture surface adhesion. Laminin was used as a positive control, as it is a well-characterized protein that naturally promotes neurite extension and neuronal adhesion, and is the ECM protein from which the IKVAV signal was derived. Comparing the material to laminin allows for a functional assessment of whether the IKVAV-functionalized fibers provide a similar or enhanced bioactive environment for neurons.

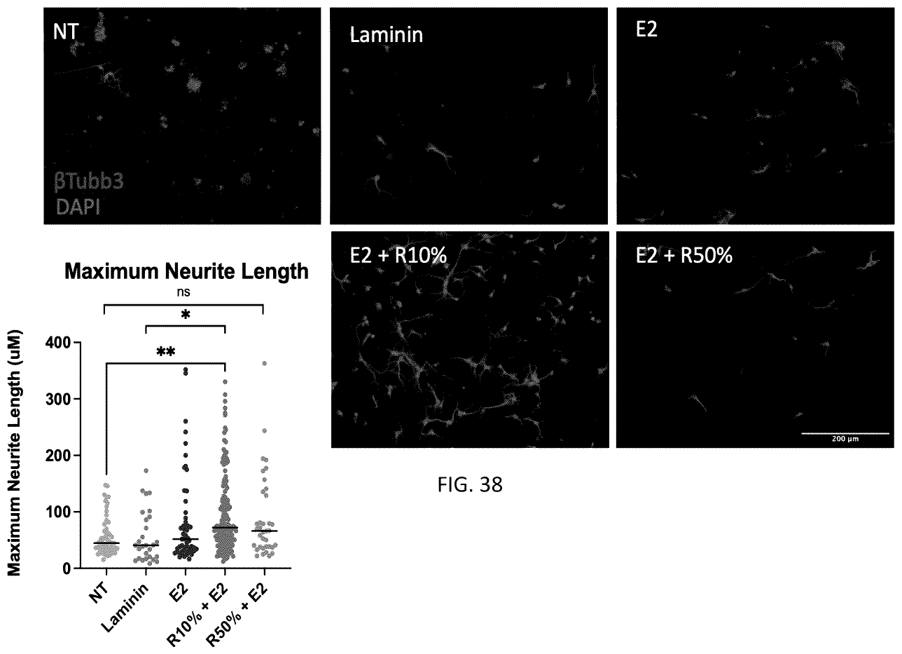

Coated samples demonstrated longer neurite outgrowth and more mature neuronal morphology compared to topical application, as the only significant differences in maximum neurite length were found in the coated samples between E2+R10% and both NT and laminin. This suggests that the slider material has a significant impact on neurons beyond just the presence of the IKVAV sequence, likely due to the increased dynamics of the R10% slider presentation. Additionally, neurons in the E2+R10% coated condition exhibited a more mature morphology, with greater dendritic extension, reinforcing its role as a bioactive scaffold. No significant differences were observed in the topically treated groups, which may be due to lower cell survival rates from dissection. In both coated and topical treatments, material aggregates were observed around the neuron bodies and dendrites in the E2+R10% and E2+R50% conditions, supporting the notion that the material acts as a scaffold to facilitate neuronal attachment and outgrowth (FIG. 38).

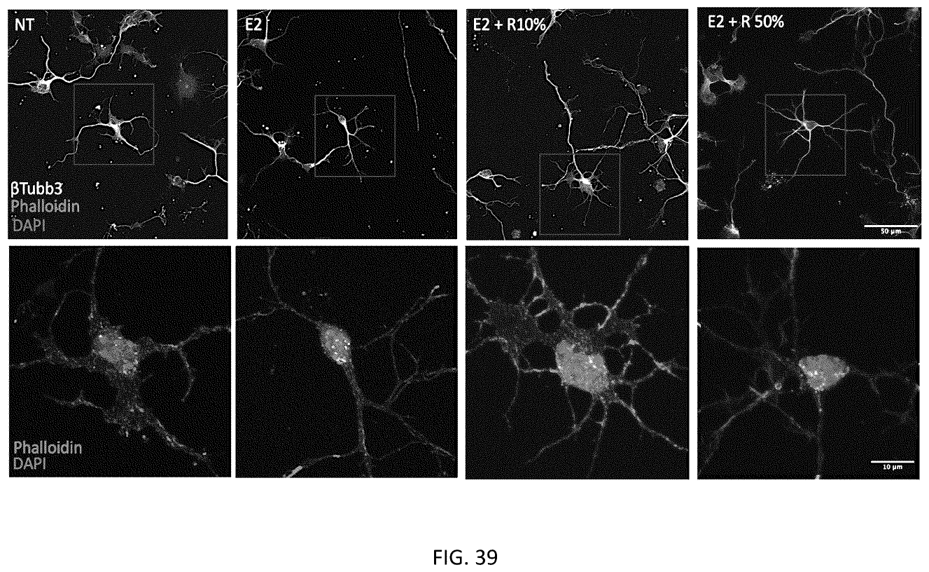

Primary Cortical Neuron Association. Further experiments were conducted to evaluate how varying concentrations of PA nanofiber materials influence integrin expression in primary cortical neurons, providing insight into how scaffold composition might modulate cell-material signaling. Although the primary focus was on integrin-mediated interactions, an unexpected but useful observation was that PA nanofibers were also stained by DAPI. This allowed clearer visualization of the fiber bundles and their spatial relationship to neurons. In the E2+R50% treatment group, bundles were seen associating closely with both the cell soma and dendrites (FIG. 39), supporting the material's potential role as a scaffold for neuronal growth. These findings offer a possible physical explanation for how bioactive signals are delivered to and retained near cells.

### Study of IKVAV Sliders on hNPCs

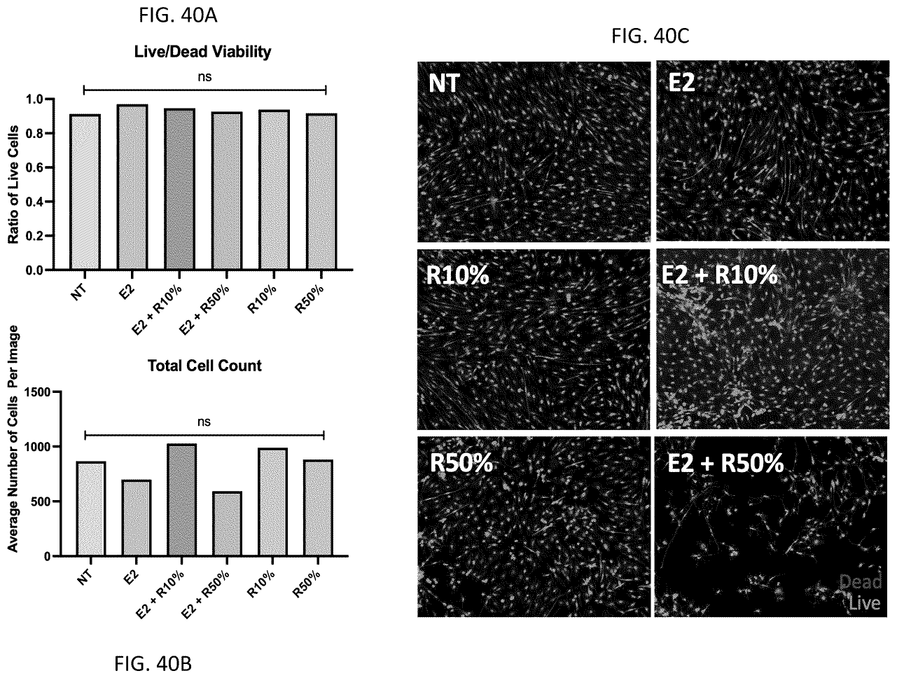

Cytotoxicity. hNPCs were used as a complementary model to assess more specific downstream biological effects of the slider-functionalized materials. A live/dead assay was performed on hNPC cultures treated with 20 μM slider material for one week. This lower concentration was selected because previous experiments indicated that hNPCs are more sensitive to PA concentrations compared to their primary counterparts. The time point was selected to maintain the culture in the undifferentiated state, while providing ample time for cell-material interaction and signaling. The assay revealed that none of the treatments have a statistically significant difference in the ratio of live/dead cells. There was also no significant difference in the number of total cells, although visually there was mildly lower cell density in the E2+R50% treatment group (FIG. 40). This could be due to the material being more effective at signaling cells to bring differentiation into neurons, which would slow the division rate of cells, leading to lower cell density but not higher death rates.

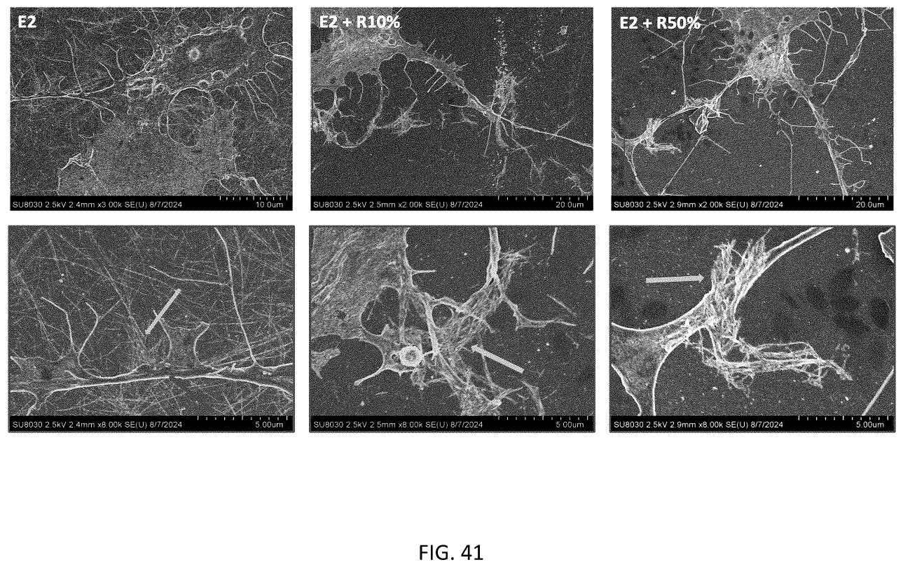

Cell Interaction with Material. To further explore the material and cell association, SEM was performed on hNPCs treated with material in solution (FIG. 41). Since the cells were allowed to adhere before treatment and were then exposed to the material for 24 hours prior to fixation, this setup allows for better interpretation of the directionality of interaction. By applying the PA material after initial cell adhesion, it becomes easier to determine whether the material preferentially binds to pre-adhered cells or whether cells migrate toward and associate with the material itself. In the E2 sample without bioactive IKVAV epitopes, the fibers formed a homogenous layer on the coverslip, with no unique interactions when cells were present. In contrast, the samples of E2 fibers with either R10% or R50% IKVAV sliders had clear associations with cells, with the dendrites of cells appearing to grow both through and over the bundles, using it like a scaffold. In the E2+R10% and E2+R50% samples, there were clear bundles of the PA nanofibers present on the coverslip.

Furthermore, on the coverslips treated with E2+R50%, all the bundles observed were associated with cells; it was impossible to find any bundles alone, even though the cell density allowed for vacant areas on the coverslip to be occupied by PA materials. It is possible that cells grow towards the IKVAV signal and use the bundles as an anchor or growth point. This provides opportunities of the IKVAV sequence on the slider to interact with cell receptors on the surface of the dendrites, promoting neuronal growth and maturation.

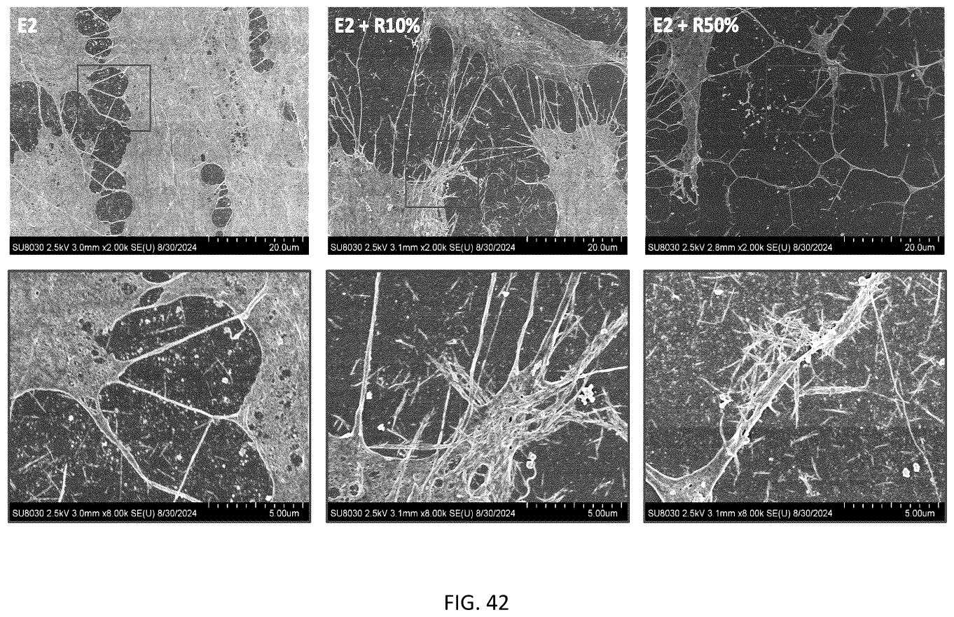

Cells Cultured With Material. To further understand the long term association between cells and PA scaffolds, hNPCs were treated until DIV8 with the same samples as previously tested. Similarly, in the samples treated with E2 PAs alone, there was a homogeneous layer of fibers on the coverslip, but no unique association between the fibers and the cells (FIG. 42). However, in the E2+R10% and E2+R50% treatments, a close association between cell dendrites and material bundles was observed. It appears that more cellular outgrowth is concentrated near bundles, and neurites frequently extend through or along the bundled structures. This suggests that the bundles continue to act as a scaffold over time, reinforcing their potential role in promoting neuronal attachment and outgrowth.

Notably, in the E2+R50% condition, more neuron-like morphology is apparent, with cells exhibiting extended dendrites and additional projections through the bundles. This observation suggests that higher concentrations of RIKVAV sliders may further enhance neuronal differentiation, maturation, and complexity.

Integrin Upregulation Visualized with Immunostaining. If the material is working to signal hNPCs, one of the first observable changes would be an upregulation of integrin, indicating recruitment of the β1-integrin receptor through IKVAV signaling. This change should be visible after a few days, so hNPCs treated on DIV1 and fixed on DIV4 were used. All hNPC experiments, including the no-treatment (NT) control, were performed on surfaces coated with poly-L-ornithine (PLO) and laminin to ensure a consistent baseline for cell adhesion. This allows the NT condition to serve as a directly comparable control, ensuring that any observed differences in integrin expression result from material treatment rather than variations in adhesion substrate.

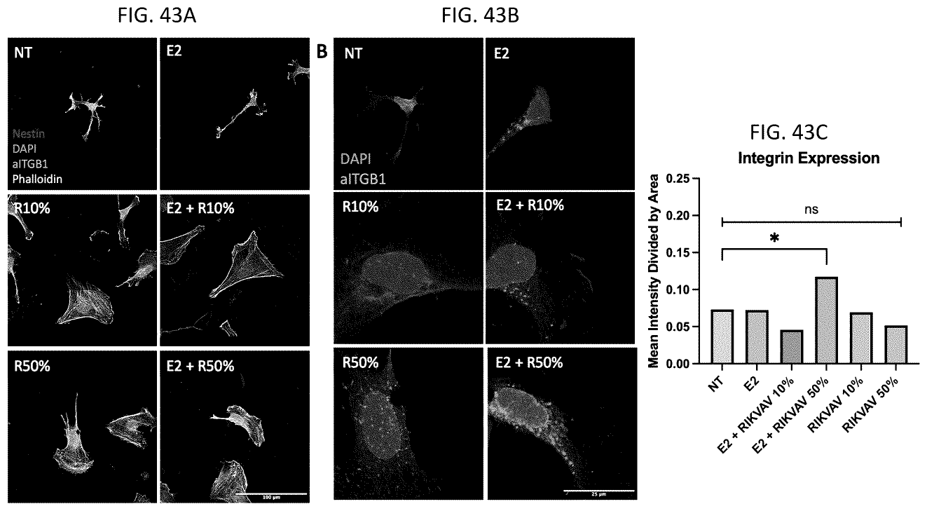

Immunostaining can be used to visualize the change in integrin, as integrin localizes on the cell surface, and when upregulated, is concentrated in adhesion sites. This concentration of integrin (ITGB) in adhesion sites is indicated by enhanced fluorescent signal when staining with an anti-ITGB1 antibody, providing strong visual upregulation of integrin. FIG. 43 shows that there is a notable increase in intensity of integrin in the E2+R50% sample. Specifically, there are more bright green ‘dots’ on the cell surface, which is strong visual confirmation of ITGB1 upregulation. Analysis of the fluorescence intensity of the aITGB1 channel showed a statistically significant difference between E2+R50% and the control. This was the only treatment that has statistical significant difference, including the samples with just fiber and just the 50% slider alone. This signifies that presentation of the IKVAV slider on E2 fibers is essential for biological activation and subsequent integrin upregulation, and is dependent on IKVAV signal concentration.

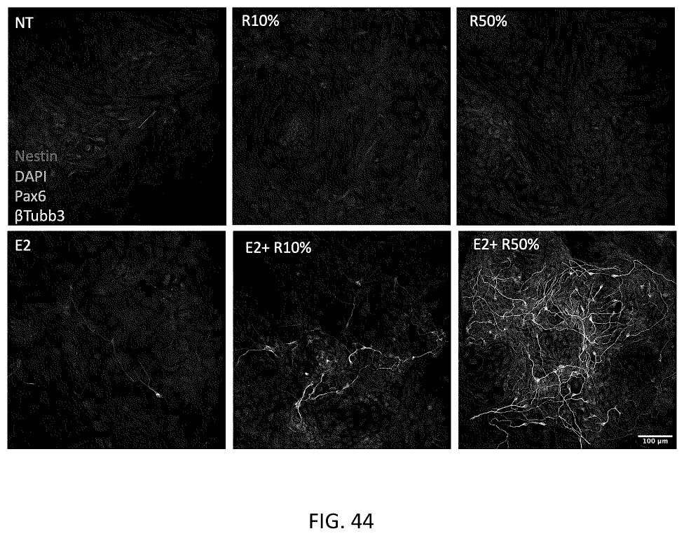

Differentiation of hNPCs Visualized with Immunostaining. To assess the differentiation of hNPCs after treatment with the material, immunostaining was performed on DIV8 cells treated with 20 μM material on DIV1. Nestin (a stem cell marker), Pax6 (a nuclear stem cell marker), and βTubb3 (a mature neuronal marker) were used to evaluate the differentiation status of the cells. In the no-treatment group, cells displayed homogeneous morphology with minimal evidence of neuronal differentiation, as indicated by sparse βTubb3 staining. Similarly, groups treated with the R10% slider, R50% slider, and E2 fibers alone exhibited comparable morphology to the no-treatment group, with a low prevalence of differentiated neurons. In contrast, cells treated with the combined material E2+R10% showed a noticeable increase in differentiated neurons compared to the no-treatment group, as evidenced by more βTubb3 positive cells. The E2+R50% treatment yielded even more striking results, with a substantially higher prevalence of differentiated neurons (FIG. 44). These neurons exhibited long axons and appeared in large clusters, indicating a more robust differentiation process.

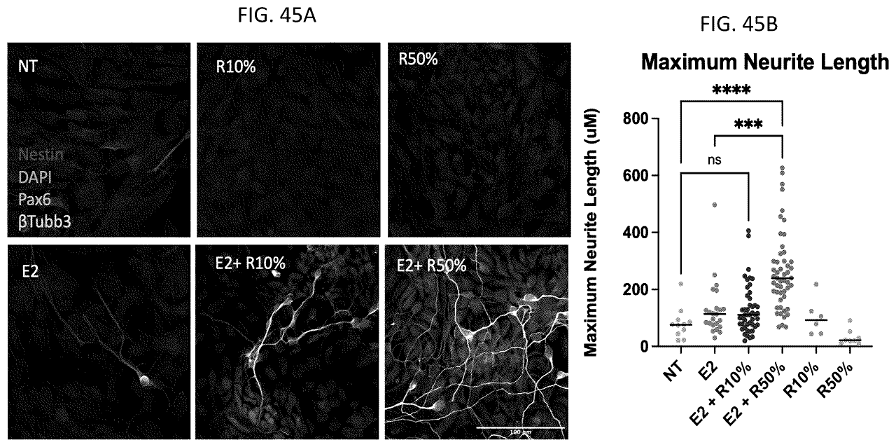

Neurite Length of Differentiated hNPCs Visualized with Immunostaining. To further characterize the differentiation and morphological changes, maximum neurite lengths were quantified using immunostaining images. Quantitative analysis revealed a significant increase in maximum neurite length for the E2+R50% treatment group compared to the no-treatment group, demonstrating the material's ability to enhance neurite extension. A significant difference was also observed between the E2 alone and E2+R50% treatment groups, highlighting the role of the R50% slider in promoting neuronal outgrowth. No significant differences were found between the no-treatment group and other treatment groups, including the R10%, R50%, and E2 treatments (FIG. 45). These results emphasize the unique ability of the E2+R50% material to promote neuronal differentiation and extensive neurite outgrowth, suggesting its strong bioactivity and potential utility in neural tissue engineering. This may be due to the high local concentration and dynamic presentation of the IKVAV epitope-supporting its strong bioactivity and potential utility in neural tissue engineering. This is also in line with the integrin upregulation of this treatment as seen in FIG. 43.

1. A system comprising:
a) a peptide amphiphile comprising a hydrophobic segment, a structural peptide segment, and a charged peptide segment; and
b) a peptide that interacts non-covalently with the peptide amphiphile.

2. The system of claim 1, wherein system comprises a nanofiber.

3. The system of claim 1, wherein the peptide comprises a biomimetic sequence and a charged sequence, wherein the charged sequence interacts non-covalently with the charged peptide segment of the peptide amphiphile.

4. The system of claim 3, wherein the charged sequence interacts electrostatically with the charged peptide segment.

5. The system of claim 4, wherein the charged peptide segment is negatively charged and wherein the charged sequence is positively charged.

6. The system of claim 1, wherein the charged sequence comprises 4 to 20 positively charged amino acids or 4 to 10 positively charged amino acids.

7. The system of claim 6, wherein the charged sequence comprises 4 to 10 lysine and/or arginine residues.

8. The system of claim 1, wherein the charged sequence comprises GRKKRRQRRRC (SEQ ID NO: 1) or a sequence having at least 80% identity to SEQ ID NO: 1.

9. The system of claim 3, wherein the biomimetic sequence comprises a growth factor mimetic sequence, a cytokine mimetic sequence, a laminin mimetic sequence, an integrin mimetic sequence, an intracellular sigma peptide (ISP) sequence, or a truncate thereof.

10. The system of claim 9, wherein the biomimetic sequence comprises an intracellular sigma peptide (ISP) sequence; a ciliary neurotrophic factor (CNTF) mimetic sequence; a vascular endothelial growth factor (VEGF) mimetic sequence, a brain-derived neurotrophic factor (BDNF) mimetic sequence, a glial cell-derived neurotrophic factor (GDNF) mimetic sequence, a fibroblast growth factor 2 (FGF-2) mimetic sequence, or a netrin-1 mimetic sequence.

11. The system of claim 10, wherein the ISP sequence comprises DSLKLSQEYESI SEQ ID NO: 2 or DMAEHMERLKANDSLKLSQEYESI (SEQ ID NO: 3).

12. The system of claim 10, wherein the CNTF mimetic sequence comprises VGDGGLFEKKL (SEQ ID NO: 4).

13. The system of claim 9, wherein the biomimetic sequence comprises the VEGF mimetic sequence KLTWQELYQLKYKGI (SEQ ID NO: 8), the BDNF mimetic sequence RKKADP (SEQ ID NO: 9), the GDNF mimetic sequence ILKNLSRSR (SEQ ID NO: 24), the FGF-2 mimetic sequence YRSRKYSSWYVALKR (SEQ ID NO: 5), the netrin-1 mimetic sequence EIDPK (SEQ ID NO: 11), the netrin-1 mimetic sequence DIDPK (SEQ ID NO: 12), RGDS (SEQ ID NO: 6), or IKVAV (SEQ ID NO: 7).

14. The system of claim 1, wherein the peptide is mobile on the peptide amphiphile.

15. The system of claim 1, comprising 1% mol to 60% mol of the peptide.

16. The system of claim 15, comprising 1%-60% mol of the peptide comprising the biomimetic sequence of SEQ ID NO: 7, 1%-10% mol of the peptide comprising the biomimetic sequence of SEQ ID NO: 4, or 5% to 10% mol of the peptide comprising the biomimetic sequence SEQ ID NO: 2 or SEQ ID NO: 3.

17. The system of claim 1, further comprising cells.

18. The system of claim 17, wherein the cells comprise neurons.

19. A method of treating an injury in a subject, comprising providing to the subject the system of claim 1.

20. The method of claim 19, wherein the injury comprises a central nervous system injury, optionally wherein the injury comprises a spinal cord injury.
